(19) United States
(12) Patent Application Publication
(10) Pub. No.: US 2009/0270447 A1
(43) Pub. Date: Oct. 29, 2009

(54) 2-Aryl-and 2-Heteroarylthiazolyl Compounds, Methods for Their Preparation and Use Thereof

(75) Inventors: Nan Zhang, Bayside, NY (US); Semiramis Ayral-Kaloustian, Tarrytown, NY (US); Thai Hiep Nguyen, Fair Lawn, NJ (US); Chuansheng Niu, Cheshire, CT (US); Edward Christian Rosfjord, Oakland, NJ (US); Ronald Suayan, Hoboken, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/412,538

(22) Filed: Mar. 27, 2009

Related U.S. Application Data
(60) Provisional application No. 61/039,873, filed on Mar. 27, 2008.

(51) Int. Cl. A61K 31/4439; C07D 471/04; A61P 35/00

(52) U.S. Cl. 514/301; 546/114

(57) ABSTRACT

The present invention discloses fused heterobicyclic 2-aryl- and 2-heteroarylthiazolyl compounds and their pharmaceutically acceptable salts and esters thereof, which are useful for inhibiting the growth of cancerous cells, inhibiting human breast carcinoma tumor growth in particular and to treat diseases or disorders associated with securin.

### CROSS-REFERENCE TO RELATED APPLICATIONS

This application claims the benefit under 35 U.S.C. §119(e) to U.S. Provisional Application Ser. No. 61/039,873, filed Mar. 27, 2008, which is hereby incorporated by reference in their entirety.

### FIELD OF THE INVENTION

The present invention relates to compounds that prevent, inhibit and/or terminate growth of transformed and cancerous cell lines during tumorigenesis. More specifically, the present invention is directed to certain fused heterobicyclic 2-substituted aryl- and heteroarylthiazolyl compounds and their pharmaceutically acceptable salts. The invention is also directed to methods for preparing fused heterobicyclic 2-aryl- and 2-heteroarylthiazolyl compounds, including their pharmaceutically acceptable salts, methods for preparing pharmaceutical compositions and formulations that include fused heterobicyclic 2-aryl- and 2-heteroarylthiazolyl compounds and methods for treating diseases associated with securin activity and inhibiting abnormal growth of certain cell types using fused heterobicyclic 2-aryl- and 2-heteroarylthiazolyl compounds.

### BACKGROUND OF THE INVENTION

Cancer is characterized by the uncontrolled rapid growth of abnormal cells, which spread to other tissues and organs through the lymphatic system or blood stream. Tumorigenesis in mammals is a multi-step process in which accumulation of genetic alterations drives the progressive transformation of normal cells to tumor cells. Genetic instability is required to generate the multiple mutations underlying cancer. An estrogen induced transforming gene securin is implicated in functional mechanisms related to cell-cycle control and tumorigenesis. It has been shown, for example, that securin is overexpressed in breast cancer and is associated with metastatic tumors, tumor spread and lymph node invasion. Anti-cancer agents are used to treat and control the growth of these cancerous cells by inhibiting, preventing and/or destroying the cancerous cells.

Certain thiazoles substituted at the 2-position are known in the prior art and their corresponding uses as a pesticide, a sedative, an anti-inflammatory or an antipyretic. For example, Japanese Patent No. JP 10-017569, discloses certain 5-N-alkyl-2-phenyl-4,5,6,7-tetrahydrothiazolo[5,4-c]pyridine compounds, wherein the phenyl group is substituted with 2-methoxy or 3-chloro and the 5-alkyl-4,5,6,7-tetrahydro[5,4-c]pyridine ring fused to the thiazole ring is not substituted, and methods for using the compounds as 5 HT3 agonists. There is a need to identify and characterize new fused heterobicyclic 2-aryl- and 2-heteroarylthiazolyl compounds that inhibit growth of tumor cells and are useful for treating diseases associated with securin. Little is known regarding how various functional groups substituted on the 2-aryl or 2-heteroaryl ring and on the heterocyclic ring fused to the thiazolyl ring, in addition to the effects of heterocyclic groups fused to the thiazolyl ring, influence structure-activity relationships (SAR). The fused heterobicyclic 2-aryl- or 2-heteroarylthiazolyl compounds of the present invention fulfill this unmet need and are useful for inhibiting the growth of cancerous cells, inhibiting human breast carcinoma tumor growth in particular and to treat diseases or disorders associated with securin activity, including elevated securin levels.

### SUMMARY OF THE INVENTION

The present invention provides fused heterobicyclic 2-aryl- or 2-heteroarylthiazolyl compounds of formula I:

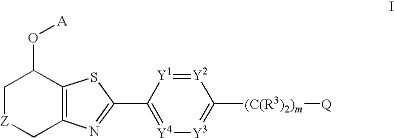

and pharmaceutically acceptable salts thereof,
wherein
A is H, C₁-C₃ alkyl, or acetyl;

### Z is NR³ or O;

### Q is —NR¹R², —NR³N(R³)₂, —NR³OR³, or —OH;

R¹ and R² are each independently H or C₁-C₃ alkyl, or R¹ and R² join together with the nitrogen atom to which each is attached, forming a 4 to 6 membered saturated heterocyclic ring comprising heteroatoms selected from 1-2 nitrogen atoms, 0-1 oxygen atom and 0-1 sulfur atom, said ring optionally substituted with one or more of R⁴;
R³ at each occurrence is independently H or C₁-C₃ alkyl;
R⁴ is C₁-C₃ alkyl, —N(R³)₂, or —OH;
Y¹, Y², Y³, and Y⁴ are the same or different, and are each independently N or CR⁵, or two R⁵ groups on adjacent carbon atoms join together, with the carbon atoms which they are bonded, to form a 9 to 10 membered bicyclic aryl ring or bicyclic heteroaryl ring, said ring comprising members selected from CR⁵ and N;
R⁵ is independently H or is independently selected from C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹, R², —C(R³)₂NR³[C(R³)₂]₂NR¹R², —CO₂R⁶, —SOR⁶, and —SO₂R⁶, where Ar is phenyl, 2-pyridyl, 3-pyridyl, 4-pyridyl, 2-thienyl, 3-thienyl, 2-furyl, or 3-furyl, optionally substituted with one or more of R⁴;
R⁶ is independently H or is independently selected from C₁-C₈ alkyl, C₂-C₈ alkenyl, and C₂-C₈ alkynyl, each optionally substituted with —NR¹R², —OR³, C₄-C₆ cycloalkyl, a saturated heterocyclic ring comprising heteroatoms selected from 0-1 nitrogen atom, 0-1 oxygen atom and 0-1 sulfur atom, or —COCH₃, said C₄-C₆ cycloalkyl, optionally substituted with R⁴; or a saturated heterocyclic ring comprising heteroatoms selected from 0-1 nitrogen atom, 0-1 oxygen atom and 0-1 sulfur atom, and optionally substituted with one or more of R⁴; and
m is 0 or 1.

The present invention also provides esters of fused heterobicyclic 2-aryl- or 2-heteroarylthiazolyl compounds of formula I.

The present invention provides a pharmaceutical composition comprising: a fused heterobicyclic 2-aryl- or 2-heteroarylthiazolyl compound of formula I, including pharmaceutically acceptable salts or esters thereof, and one or more pharmaceutically acceptable carriers.

The present invention also includes a method for preparing a fused heterobicyclic 2-aryl- or 2-heteroarylthiazolyl compound of formula I and pharmaceutically acceptable salts thereof, comprising the steps of:

(a) reacting a substituted arylnitrile compound of formula 1:

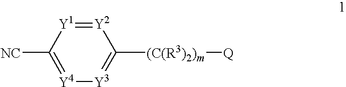

with phosphorous pentasulfide, thereby forming a substituted thioamide compound of formula 2:

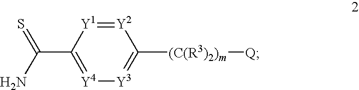

(b) reacting the substituted thioamide compound of formula 2 with tert-butyl 5-oxo-7-oxa-3-azabicyclo[4.1.0]heptane-3-carboxylate, thereby forming a protected compound of formula 3:

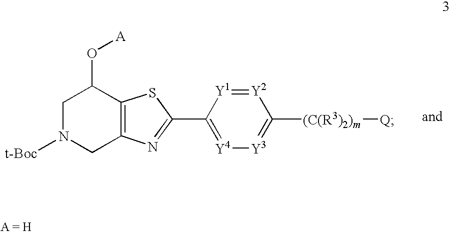

(c) treating the protected compound of formula 3 formed in step (b) with an acid, thereby forming the compound of formula I or a pharmaceutically acceptable salt thereof, wherein A, Q, Y¹-Y⁴, R³, and m are defined above.

The present invention also includes a method for preparing a fused heterobicyclic 2-aryl- or 2-heteroarylthiazolyl compound of formula I and pharmaceutically acceptable salts thereof, comprising the steps of:

(a) reacting a substituted arylnitrile compound of formula 1 with phosphorous pentasulfide, thereby forming a substituted thioamide compound of formula 2, as shown above;
(b) reacting the substituted thioamide compound of formula 2 formed in step (a) with 1,3-dibromoacetone, thereby forming a bromoalkylthiazole compound of formula 4:

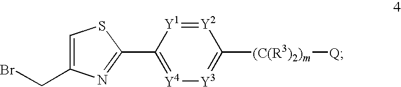

(c) brominating the bromoalkylthiazole compound of formula 4 formed in step (b), thereby forming an intermediate dibromothiazole compound of formula 5:

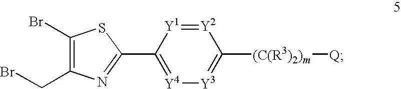

(d) reacting the intermediate compound dibromothiazole compound of formula 5 formed in step (c) with a compound of formula 6:

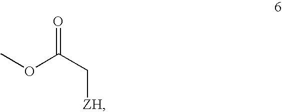

where Z is NH,
thereby forming an intermediate thiazole compound of formula 7:

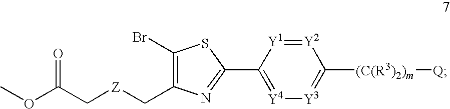

(e) treating the intermediate thiazole compound of formula 7 formed in step (d) with t-Boc₂O to produce an intermediate protected thiazole compound of formula 8:

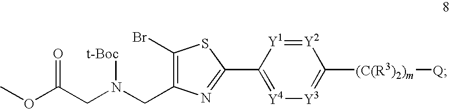

(f) cyclizing the intermediate protected thiazole compound of formula 8 formed in step (e) with butyl lithium, thereby forming an intermediate protected, fused heterobicyclic thiazolyl compound of formula 9:

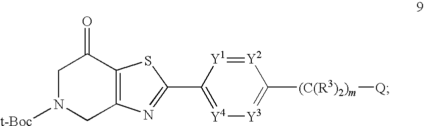

(g) treating the intermediate protected, fused heterobicyclic thiazolyl compound of formula 9 formed in step (f) with a reducing agent (sodium borohydride), thereby forming an intermediate protected, fused heterobicyclic thiazolyl compound of formula 10:

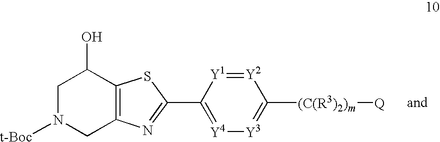

(h) treating the intermediate protected, fused heterobicyclic thiazolyl compound of formula 10 formed in step (g) with a deprotecting agent, thereby forming the fused heterobicyclic 2-aryl- or 2-heteroarylthiazolyl compound of formula I or a pharmaceutically acceptable salt thereof, wherein Q, Y¹-Y⁴, R³, and m are defined above.

The present invention also includes a method for preparing a fused heterobicyclic 2-aryl- or 2-heteroarylthiazolyl compound of formula I and pharmaceutically acceptable salts thereof, comprising the steps of:

(a) reacting a substituted arylnitrile compound of formula 1 with phosphorous pentasulfide, thereby forming a substituted thioamide compound of formula 2, as shown above;
(b) reacting the substituted thioamide compound of formula 2 formed in step (a) with 1,3-dibromoacetone, thereby forming a bromoalkylthiazole compound of formula 4, as shown above;
(c) brominating the bromoalkylthiazole compound of formula 4 formed in step (b), thereby forming an intermediate dibromothiazole compound of formula 5, as shown above;
(d) reacting the intermediate compound dibromothiazole compound of formula 5 formed in step (c) with a compound of formula 6:

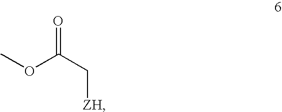

where Z is O or N(C₁-C₃ alkyl)
thereby forming an intermediate thiazole compound of formula 7:

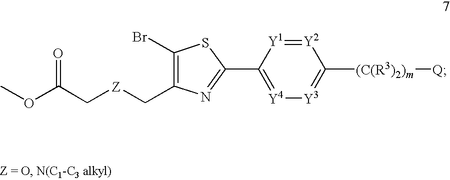

(e) cyclizing the intermediate thiazole compound of formula 7 formed in step (d) with butyl lithium, thereby forming an intermediate fused heterobicyclic thiazolyl compound of formula 9:

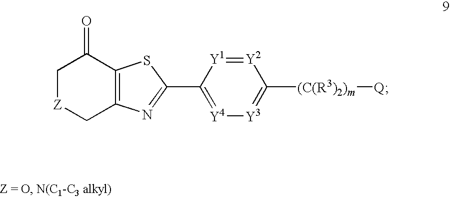

(f) treating the intermediate fused heterobicyclic thiazolyl compound of formula 9 formed in step (e) with a reducing agent (sodium borohydride), thereby forming a fused heterobicyclic 2-aryl- or 2-heteroarylthiazolyl compound of formula I

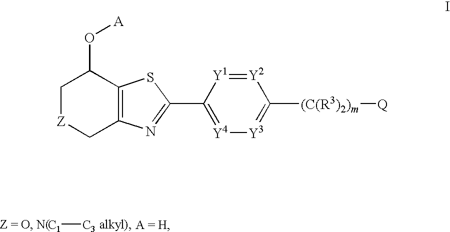

or a pharmaceutically acceptable salt thereof, wherein Q, Y¹-Y⁴, R³, and m are defined above.

Accordingly, the invention provides a method of inhibiting the growth of cancerous cells or tumors comprising the step of: administering to a patient in need a pharmaceutically effective amount of a fused heterobicyclic 2-aryl- or 2-heteroarylthiazolyl compound of formula 1. The invention also provides a method of treating a disease associated with securin activity comprising the step of administering to a subject in need a therapeutically effective amount of one or more fused heterobicyclic 2-aryl- or 2-heteroarylthiazolyl compounds of formula I and pharmaceutically acceptable salts thereof. In one embodiment, securin activity is associated with a human breast carcinoma. In other embodiments, securin activity is associated with one or more cancers selected from: leukemia, brain cancer, lung cancer, colon cancer, thyroid cancer, ovarian cancer, renal cancer and prostate cancer. Other embodiments of the invention are found in the following detailed description.

### BRIEF DESCRIPTION OF THE DRAWINGS

FIG. 1 is a plot comparing the effectiveness of Examples 5-7 versus Vincristine at controlling tumor growth.

### DETAILED DESCRIPTION OF THE INVENTION

The term “alkyl” refers to the radical of saturated aliphatic groups of 1 to 8 carbon atoms, including straight-chain alkyl groups, branched-chain alkyl groups, cycloalkyl (alicyclic) groups, alkyl substituted cycloalkyl groups, and cycloalkyl substituted alkyl groups. In one embodiment, a straight chain or branched chain alkyl has 6 or fewer carbon atoms in its backbone. The term “alkyl” can be used alone or as part of a chemical name, such as “alkylamine”. The terms “alkenyl” and “alkynyl” refer to unsaturated aliphatic groups analogous in length and possible substitution to the alkyls described above, but which contain at least one double or triple carbon-carbon bond, respectively.

The term “aryl”, as used herein, whether used alone or as part of another group, is defined as a substituted or unsubstituted aromatic hydrocarbon ring group having 6 to about 50 carbon atoms (unless explicitly specified otherwise) with from about 6 to about 10 atoms being preferred. The “aryl” group can have a single ring or multiple condensed rings. The term “aryl” includes, but is not limited to phenyl, α-naphthyl, β-naphthyl, biphenyl, anthryl, tetrahydronaphthyl, fluorenyl, indanyl, biphenylenyl, and acenaphthenyl. Specifically included within the definition of “aryl” are those aromatic groups that are optionally substituted. For example, in representative embodiments of the present invention, the, “aryl” groups are optionally substituted with from 1 to 5 substituents selected from: H, C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², C(R³)₂NR³[C(R³)₂]₂NR¹R², —CO₂R⁶, —SOR⁶, or —SO₂R⁶, where Ar is phenyl, 2-pyridyl, 3-pyridyl, 4-pyridyl, 2-thienyl, 3-thienyl, 2-furyl, or 3-furyl, optionally substituted with C₁-C₃ alkyl, —N(R³)₂, and —OH.

The term “heteroaryl” as used herein is defined as a substituted or unsubstituted aromatic heterocyclic ring system (monocyclic or bicyclic). Heteroaryl groups can have, for example, from about 3 to about 50 carbon atoms and 1-8 heteroatoms (unless explicitly specified otherwise) with from about 4 to about 10 carbon atoms and 1-4 heteroatoms being preferred. In some embodiments, heteroaryl groups are aromatic heterocyclic rings systems having about 4 to about 14 ring atoms including carbon atoms and 1, 2, 3, or 4 heteroatoms selected from oxygen, nitrogen or sulfur. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Specifically included within the definition of “heteroaryl” are those aromatic groups that are optionally substituted. Accordingly, the heteroaryl groups described herein include both unsubstituted or substituted groups. Suitable examples of monocyclic and bicyclic heteroaryl groups are selected from: furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. In representative embodiments of the present invention, the, “heteroaryl” groups are optionally substituted with 1 to 5 substituents selected from: H, C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, or —SO₂R⁶, where Ar is phenyl, 2-pyridyl, 3-pyridyl, 4-pyridyl, 2-thienyl, 3-thienyl, 2-furyl, or 3-furyl, optionally substituted with C₁-C₃ alkyl, —N(R³)₂, and —OH.

The term “heterocycle”, as used herein, whether used alone or as part of another group, refers to a stable 3 to about 10-member ring containing carbons atoms and from 1 to 4 heteroatoms selected from the group consisting of nitrogen, oxygen, and sulfur. A heterocycle of this invention can be either a monocyclic or bicyclic ring system, and can be either saturated, unsaturated, or partially saturated. A heterocycle can be optionally fused to a phenyl ring or a thiazole ring. The term “heterobicyclic ring” refers to a bicyclic ring system comprising at least one heterocycle. Suitable examples of heterocycles include, but are not limited to, aziridinyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl. Preferred heterocycle moieties include: (a) 6-membered saturated, partially unsaturated, or unsaturated heterocycles containing 1-2 nitrogens, optionally fused to a phenyl ring; (b) 5-membered saturated, partially saturated, or unsaturated heterocycles containing 1-3 nitrogen, oxygen, or sulfur atoms, optionally fused to a phenyl ring; (c) saturated, partially unsaturated, or unsaturated bicyclic heterocycles containing 1-4 nitrogen, oxygen, or sulfur atoms; (d) carbazole, dibenzofuran, and dibenzothiophene. Specifically included in the definition of “heterocycle” are those heterocycles that are optionally substituted with one to four substituents selected from: H, C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹, R², —C(R³)₂NR³[C(R³)₂]₂NR¹, R², —CO₂R⁶, —SOR⁶, or —SO₂R⁶, where Ar is phenyl, 2-pyridyl, 3-pyridyl, 4-pyridyl, 2-thienyl, 3-thienyl, 2-furyl, or 3-furyl, optionally substituted with C₁-C₃ alkyl, —N(R³)₂, and —OH.

The term “halogen” refers to an atom of fluorine, chlorine, bromine, or iodine. As used herein, the term “substituted” is contemplated to include all permissible substituents of organic compounds. Typically, suitable substituents of organic compounds include acyclic and cyclic, branched and unbranched, carbocyclic and heterocyclic, aromatic and nonaromatic substituents of organic compounds as well as inorganic substituents such as halogen or amino. The substituents can be one or more and the same or different for appropriate organic compounds. For purposes of this invention, the heteroatoms such as nitrogen may have hydrogen substituents, halogen substituents and/or any suitable substituents of organic compounds described herein which satisfy the valencies of the heteroatoms. This invention is not intended to be limited in any manner by the suitable substituents of organic compounds.

Accordingly, present invention provides fused heterobicyclic 2-aryl- or 2-heteroarylthiazolyl compounds of formula I:

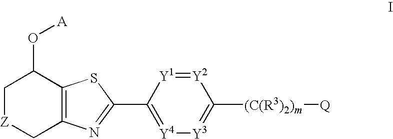

and pharmaceutically acceptable salts thereof, wherein A, Q, Z, Y¹-Y⁴, R³ and m are defined above.

Suitable examples of compounds of the invention include, but are not limited to, 2-aryl-4,5,6,7-tetrahydrothiazolo[4,5-c]pyridin-7-ol, 2-aryl-6,7-dihydrothiazolo[3,4-d]pyran-7-ol and substituted derivative compounds thereof. According to one embodiment, the N- or O-containing heterocyclic ring fused to the thiazolyl ring at the 1,3-position is substituted by functional groups selected from —OH, C₁-C₃ alkoxy, and O-acetyl.

According to one embodiment, the ring formed by Y¹, Y², Y³, and Y⁴ that is covalently bonded to the thiazole ring at the 2-position, is an aryl or heteroaryl ring of 6 atoms selected from: phenyl, pyridinyl, pyrimidinyl, and pyrazinyl. According to a separate embodiment, Y¹ and Y² or Y³ and Y⁴ are CR⁵, the two R⁵ groups on adjacent carbon atoms join together, with the carbon atoms which they are bonded, to form a 9 to 10 membered bicyclic aryl ring or bicyclic heteroaryl ring, said ring selected from: napthyl, indenyl, indolyl, benzoxazolyl, benzothiazolyl, benzofuranyl, benzisoxazolyl, benzimidazolyl, N-methylbenzimidazolyl, azabenzimidazolyl, indazolyl, quinazolinyl, quinolinyl, and isoquinolinyl.

According to one embodiment, a phenyl group is covalently bonded to the fused heterobicyclic thiazolyl ring at the 2-position of the thiazolyl ring. The phenyl group is substituted with 1 to 4 functional groups that are selected from: C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², C(R³)₂NR³[C(R³)₂]₂NR¹R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

According to a separate embodiment, a pyridinyl group is covalently bonded to the fused heterobicyclic thiazolyl ring at the 2-position of the thiazolyl ring. The pyridinyl group can be substituted at positions 2-5. The pyridinyl group is substituted with 1 to 3 functional groups that are selected from C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹, R², —C(R³)₂NR³[C(R³)₂]₂NR¹, R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

According to a separate embodiment, an indazolyl group is covalently bonded to the fused heterobicyclic thiazolyl ring at the 2-position of the thiazolyl ring. The indazolyl group is substituted with 1 to 3 functional groups at any acceptable position of the indazolyl ring, including the N-atom of the indazolyl ring, that are selected from C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

According to one embodiment, m is o, Z is NH, A is H and a phenyl group is covalently bonded to the fused heterobicyclic thiazolyl ring at the 2-position of the thiazolyl ring. The phenyl group is substituted with 1 to 4 functional groups that are selected from: C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

According to a separate embodiment, m is o, Z is C₁-C₃ alkylN, A is H and a phenyl group is covalently bonded to the fused heterobicyclic thiazolyl ring at the 2-position of the thiazolyl ring. The phenyl group is substituted with 1 to 4 functional groups that are selected from: C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

According to a separate embodiment, m is o, Z is O, A is H and a phenyl group is covalently bonded to the fused heterobicyclic thiazolyl ring at the 2-position of the thiazolyl ring. The phenyl group is substituted with 1 to 4 functional groups that are selected from: C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

Suitable examples of fused heterobicyclic 2-aryl- or 2-heteroarylthiazolyl compounds of formula I include, but are not limited to, compounds selected from:

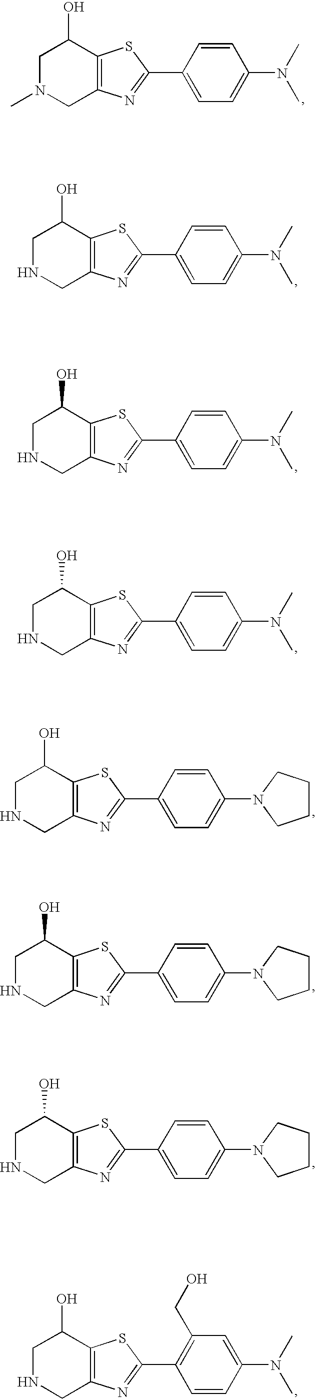

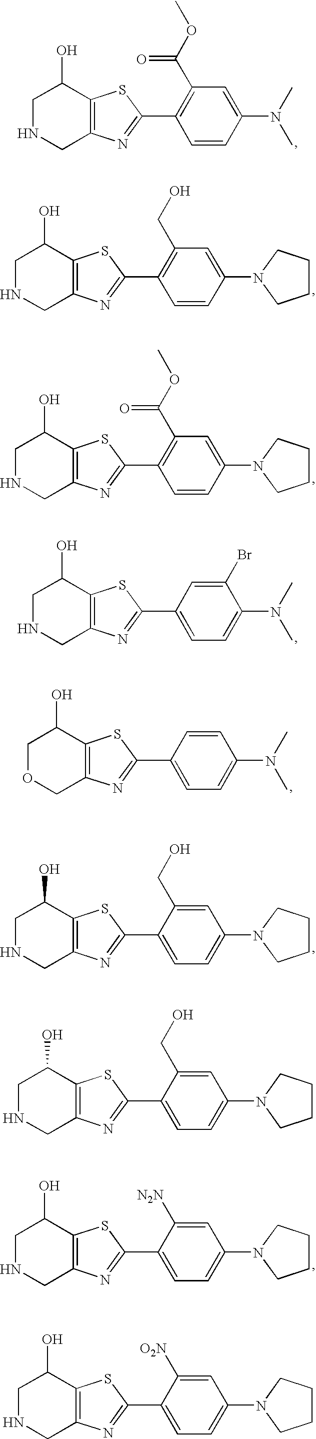

and pharmaceutically acceptable salts thereof.

Compound names corresponding to the structures of fused heterobicyclic 2-aryl- or 2-heteroarylthiazolyl compounds of formula I include, but are not limited to, compounds selected from: 2-(4-dimethylamino-phenyl)-5-methyl-4,5,6,7-tetrahydro[1,3]thiazolo[4,5-c]pyridine-7-ol, (R,S)-2-(4-dimethylamino-phenyl)-4,5,6,7-tetrahydro[1,3]thiazolo[4,5-c]pyridin-7-ol, (R)-2-(4-dimethylamino-phenyl)-4,5,6,7-tetrahydro[1,3]thiazolo[4,5-c]pyridin-7-ol, (S)-2-(4-dimethylamino-phenyl)-4,5,6,7-tetrahydro[1,3]thiazolo[4,5-c]pyridin-7-ol, (7R,S)-2-(4-pyrrolidin-1-yl-phenyl)-4,5,6,7-tetrahydro[1,3]thiazolo[4,5-c]pyridin-7-ol, (7R)-2-(4-pyrrolidin-1-yl-phenyl)-4,5,6,7-tetrahydro[1,3]thiazolo[4,5-c]pyridin-7-ol, (7S)-2-(4-pyrrolidin-1-yl-phenyl)-4,5,6,7-tetrahydro[1,3]thiazolo[4,5-c]pyridin-7-ol, 2-(2-hydroxymethyl-4-dimethylamino-phenyl)-4,5,6,7-tetrahydro[1,3]thiazolo[4,5-c]pyridine-7-ol, methyl 5-(dimethylamino)-2-(7-hydroxy-4,5,6,7-tetrahydro[1,3]thiazolo[4,5-c]pyridine-2-yl)benzoate, (7R,7S)-2-(2-hydroxymethyl-4-pyrrolidin-1-yl-phenyl)-4,5,6,7-tetrahydro[1,3]thiazolo[4,5-c]pyridin-7-ol, (7R)-2-(2-hydroxymethyl-4-pyrrolidin-1-yl-phenyl)-4,5,6,7-tetrahydro[1,3]thiazolo[4,5-c]pyridin-7-ol, (7R,7S)-2-(2-hydroxymethyl-4-pyrrolidin-1-yl-phenyl)-4,5,6,7-tetrahydro[1,3]thiazolo[4,5-c]pyridin-7-ol, methyl 5-(4-pyrrolidin-1-yl)-2-(7-hydroxy-4,5,6,7-tetrahydro[1,3]thiazolo[4,5-c]pyridin-2-yl)benzoate, 2-(2-amino-4-pyrrolidin-1-yl-phenyl)-4,5,6,7-tetrahydro[1,3]thiazolo[4,5-c]pyridin-7-ol, 2-(2-nitro-4-pyrrolidin-1-yl-phenyl)-4,5,6,7-tetrahydro[1,3]thiazolo[4,5-c]pyridin-7-ol, 2-[3-bromo-4-(dimethylamino)phenyl]-6,7-dihydro-4H-pyrano[3,4-d][1,3]thiazol-7-ol, 2-[4-(dimethylamino)phenyl]-6,7-dihydro-4H-pyrano[3,4-d][1,3]thiazol-7-ol and pharmaceutically acceptable salts thereof.

Where present, fused heterobicyclic 2-aryl- or 2-heteroarylthiazolyl compounds of formula I and corresponding pharmaceutically acceptable salts or esters thereof include isomers either individually or as a mixture, such as enantiomers, diastereomers, and positional isomers. “Pharmaceutically acceptable salts and esters” refers to salts and esters that are pharmaceutically acceptable and have the desired pharmacological properties. Such salts include, for example, salts that can be formed where acidic protons present in the compounds are capable of reacting with inorganic or organic bases. Suitable inorganic salts include, for example, those formed with the alkali metals or alkaline earth metals, e.g. sodium and potassium, magnesium, calcium, and aluminum. Suitable organic salts include, for example, those formed with organic bases such as the amine bases, e.g. ethanolamine, diethanolamine, triethanolamine, tromethamine, N-methylglucamine, and the like. Pharmaceutically acceptable salts can also include acid addition salts formed from the reaction of basic moieties, such as amines, in the parent compound with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid).

Pharmaceutically acceptable esters include esters formed from carboxy, sulfonyloxy, and phosphonoxy groups present in the compounds, e.g. C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention can be present in more than one stereoisomeric form, and the naming of such compounds is intended to include all single stereoisomers and all mixtures (whether racemic or otherwise) of such stereoisomers.

Pharmaceutically acceptable salts of fused heterobicyclic 2-aryl- or 2-heteroarylthiazolyl compounds of formula I with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acids: acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic functional group. Other suitable examples of pharmaceutically acceptable salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucaronate; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts. The compounds can also be used in the form of esters, carbamates and other conventional ester forms, also reffered to herein as prodrug forms, which when administered in such form, convert to the active moiety in-vivo. Exemplary ester forms of the compounds of this invention include, but are not limited to, straight chain alkyl esters having from 1 to 6 carbon atoms or branched chain alkyl groups containing 1 to 6 carbon atoms, including methyl, ethyl, propyl, butyl, 2-methylpropyl and 1,1-dimethylethyl esters, cycloalkyl esters, alkylaryl esters, benzyl esters, and the like.

The fused heterobicyclic 2-aryl- or 2-heteroarylthiazolyl compounds of formula I are prepared from: (a) commercially available starting materials (b) known starting materials which may be prepared as described in literature procedures or (c) new intermediates described in the schemes and experimental procedures herein. Reactions are performed in a solvent appropriate to the reagents and materials employed and suitable for the transformation being effected. It is understood by those skilled in the art of organic synthesis that the various functionalities present on the molecule is consistent with the chemical transformation proposed.

One method for preparing a fused heterobicyclic 2-aryl- or 2-heteroarylthiazolyl compound of formula I is to react an appropriately substituted arylnitrile compound of formula 1 with phosphorous pentasulfide, which yields the corresponding substituted thioamide compound of formula 2. The resulting substituted thioamide compound of formula 2 is then treated with tert-butyl 5-oxo-7-3-azabicyclo[4.1.0]heptane-3-carboxylate, which forms a t-Boc protected compound of formula 3. The t-Boc protected compound of formula 3 is treated with a deprotecting agent in the form of an acid (e.g. HCl), thereby forming the fused heterobicyclic 2-aryl- or 2-heteroarylthiazolyl compound of formula I or its pharmaceutically acceptable salt thereof. Any suitable deprotecting agent or acid is usefully employed in accordance with the invention. The sequence of reactions is summarized in Scheme 1.

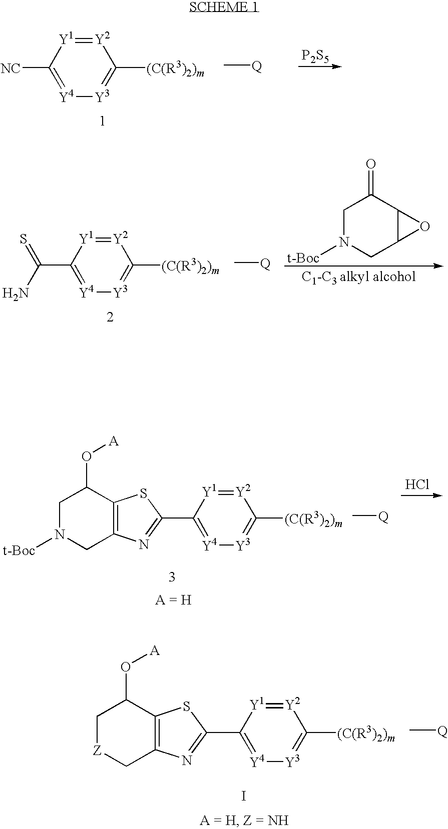

According to one embodiment, after forming the t-Boc protected compound of formula 3, the protected compound is reacted with a C₁-C₃ alkyl halide in the presence of a base. The modified t-Boc protected compound of formula 3 is then treated with a deprotecting agent, such as an acid (e.g. HCl), to remove the protecting group. The resulting compound is the alkoxy substituted, fused heterobicyclic 2-aryl- or 2-heteroarylthiazolyl compound of formula I, or its pharmaceutically acceptable salt thereof, as depicted in Scheme 2.

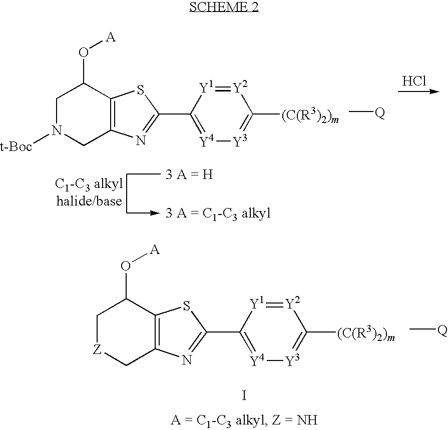

In a separate embodiment, the t-Boc protected compound may be reacted with an acetic anhydride in the presence of a base and the modified t-Boc protected compound of formula 3 is then treated with a deprotecting agent, such as an acid (e.g. HCl), to remove the protecting group. This forms the desired derivative, an acetyl ester of the fused heterobicyclic 2-aryl- or 2-heteroarylthiazolyl compound of formula 1, or its pharmaceutically acceptable salt thereof, as depicted in Scheme 3.

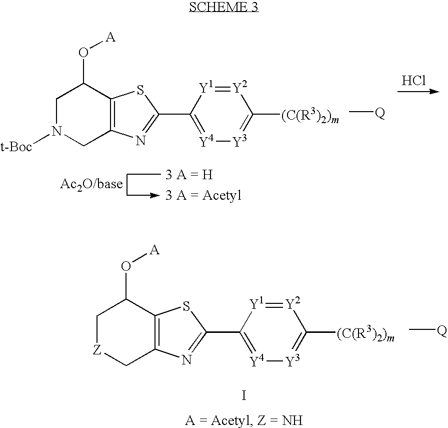

Another method for preparing a fused heterobicyclic 2-aryl- or 2-heteroarylthiazolyl compound of formula I and pharmaceutically acceptable salts thereof, as summarized in Scheme 4, comprises the steps of:

(a) reacting a substituted arylnitrile compound of formula 1 with phosphorous pentasulfide, thereby forming a substituted thioamide compound of formula 2;
(b) reacting the substituted thioamide compound of formula 2 formed in step (a) with 1,3-dibromoacetone, thereby forming a bromoalkylthiazole compound of formula 4;
(c) brominating the bromoalkylthiazole compound of formula 4 formed in step (b), thereby forming an intermediate dibromothiazole compound of formula 5;
(d) reacting the intermediate compound dibromothiazole compound of formula 5 formed in step (c) with a compound of formula 6, thereby forming an intermediate thiazole compound of formula 7;
(e) treating the intermediate thiazole compound of formula 7 formed in step (d) with t-Boc₂O to produce a protected intermediate thiazole compound of formula 8;
(f) cyclizing the protected intermediate thiazole compound of formula 8 formed in step (e) with butyl lithium, thereby forming a protected, fused heterobicyclic thiazole intermediate compound of formula 9;
(g) treating the protected, fused heterobicyclic thiazole intermediate compound of formula 9 formed in step (f) with a reducing agent (sodium borohydride), thereby forming a protected, fused heterobicyclic thiazole intermediate compound of formula 10; and
(h) treating the protected, fused heterobicyclic thiazole intermediate compound of formula 10 formed in step (g) with a deprotecting agent, thereby forming the fused heterobicyclic 2-aryl- or 2-heteroarylthiazolyl compound of formula I or a pharmaceutically acceptable salt thereof, wherein A, Z is NH, Q, Y¹-Y⁴, R³, and m are defined above. The method is illustrated in Scheme 4.

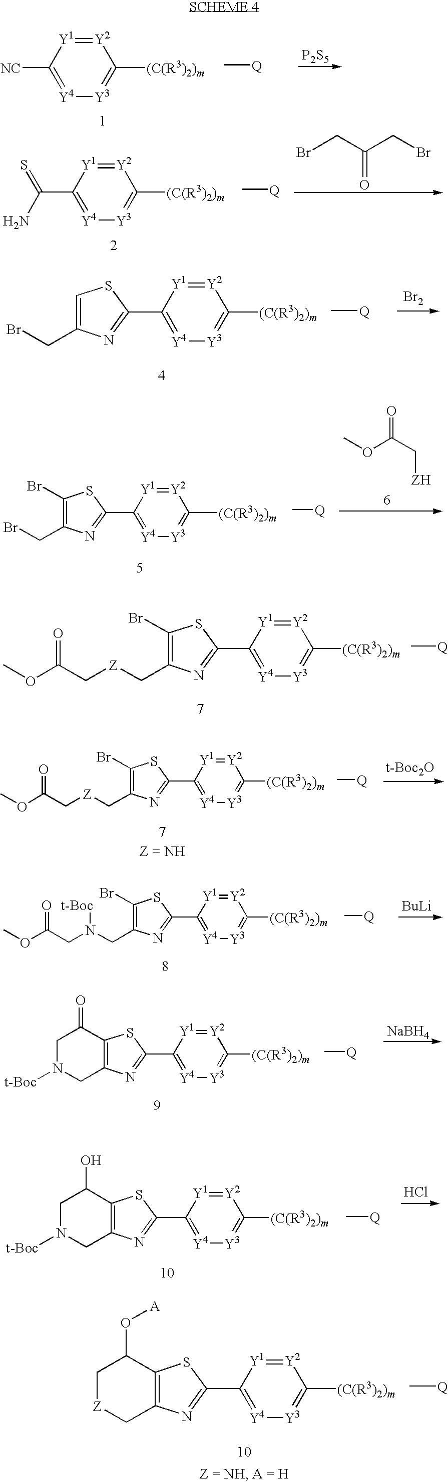

According to one embodiment, after forming the t-Boc protected compound of formula 10, the protected compound may be reacted with a C₁-C₃ alkyl halide in the presence of a base. The modified or derivative t-Boc protected compound of formula 10 is then treated with a deprotecting agent, such as an acid (e.g. HCl), to remove the protecting group. The resulting product is the alkoxy substituted, fused heterobicyclic 2-aryl- or 2-heteroarylthiazolyl compound of formula 1, or its pharmaceutically acceptable salt thereof, as shown in Scheme 5.

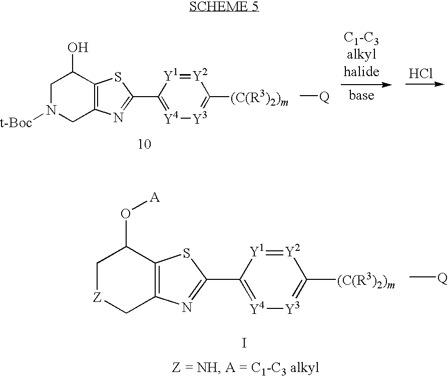

According to a separate embodiment, the t-Boc protected compound may be reacted with an acetic anhydride in the presence of a base after the protected compound of formula 10 is formed. A deprotecting agent, such as an acid (e.g. HCl), is then used to remove the protecting group from the modified t-Boc protected compound of formula 10. The resulting product is or its pharmaceutically acceptable salt thereof. This forms the desired derivative, an acetyl ester of the fused heterobicyclic 2-aryl- or 2-heteroarylthiazolyl compound of formula I, or its pharmaceutically acceptable salt thereof, as depicted in Scheme 6.

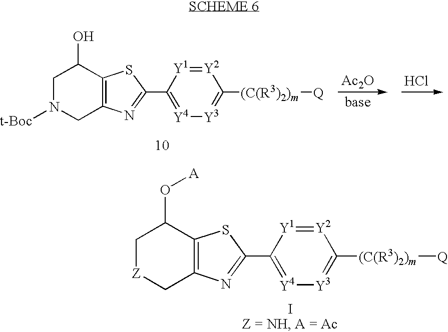

In another method for preparing a fused heterobicyclic 2-aryl- or 2-heteroarylthiazolyl compound of formula I and pharmaceutically acceptable salts thereof, the compound of formula 7 is produced by the method described above and summarized in Scheme 4, and comprises additional steps of:

(e) cyclizing the intermediate thiazole compound of formula 7 formed in step (d) with butyl lithium, thereby forming a fused heterobicyclic thiazole intermediate compound of formula 9; and
(f) treating the fused heterobicyclic thiazole intermediate compound of formula 9 formed in step (e) with a reducing agent (sodium borohydride), thereby forming a fused heterobicyclic 2-aryl- or 2-heteroarylthiazolyl compound of formula I or a pharmaceutically acceptable salt thereof, wherein Q, Y¹-Y⁴, R³, and m are defined above and summarized in Scheme 7.

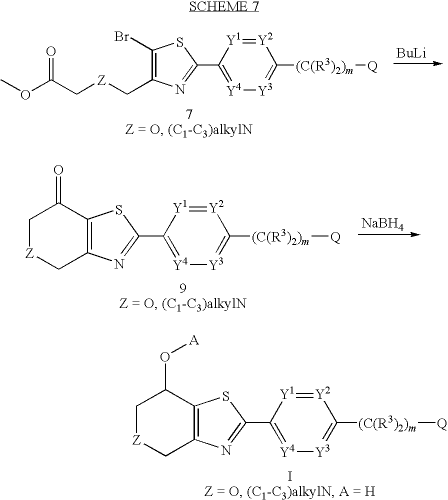

According to separate embodiments, corresponding alkoxy substituted and ester forms of the fused heterobicyclic 2-aryl- or 2-heteroarylthiazolyl compound of formula I, or pharmaceutically acceptable salts thereof, are summarized in Scheme 8.

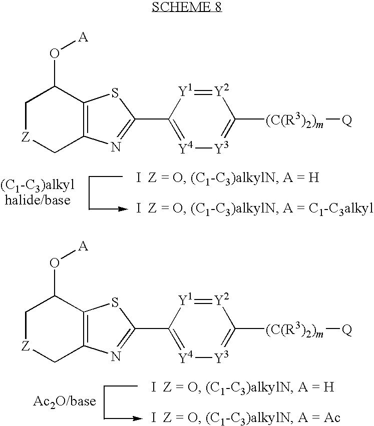

In the synthesis reactions summarized in Schemes 5-8, a base is used as a catalyst. Suitable examples of bases include, but are not limited to, 4-dimethylamino pyridine, triethylamine, pyridine, sodium carbonate, potassium carbonate, potassium hydroxide, and mixtures thereof.

The present invention accordingly provides a pharmaceutical composition, which comprises an effective amount of a fused heterobicyclic 2-aryl- or 2-heteroarylthiazolyl compound of the present invention in combination or association with a pharmaceutically acceptable carrier. Suitable examples of pharmaceutical carriers used in accordance with the present invention include, but are not limited to, excipients, diluents, fillers, disintegrants, lubricants and other agents that can function as a carrier. The term “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. Pharmaceutical compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable. As used herein, the term “effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

The term “treating” or “treatment” refers to any indicia of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” or “treatment of a securin related disorder” includes preventing the onset of symptoms in a subject that may be predisposed to a securin related disorder but does not yet experience or exhibit symptoms of the disorder (prophylactic treatment), inhibiting the symptoms of the disorder (slowing or arresting its development), providing relief from the symptoms or side-effects of the disorder (including palliative treatment), and/or relieving the symptoms of the disorder (causing regression). Accordingly, the term “treating” includes the administration of the compounds or agents of the present invention to a subject to prevent or delay, to alleviate, or to arrest or inhibit development of the symptoms or conditions associated with securin related disorders, e.g., tumor growth associated with cancer. A skilled medical practitioner will know how to use standard methods to determine whether a patient is suffering from a disease associated with enhanced levels and/or activity of securin, e.g., by examining the patient and determining whether the patient is suffering from a disease known to be associated with elevated securin levels or activity or by assaying for securin levels in blood plasma or tissue of the individual suspected of suffering from a securin related disease and comparing securin levels in the blood plasma or tissue of the individual suspected of suffering from a securin related disease to securin levels in the blood plasma or tissue of a healthy individual. Increased securin levels are indicative of disease. Accordingly, the present invention provides, inter alia, methods of administering a compound of the present invention to a subject and determining levels of securin in the subject. The level of securin in the subject can be determined before and/or after administration of the compound.

In healthy individuals, securin is found at low levels in the plasma, but it is elevated in many securin related disorders, including, for example, breast cancer (J. A. Bernal, et al. Nature Genetics, Vol. 32, pp. 306-311, 2002 and S. Ogbagabriel, et al. Mod. Path. Vol. 18, pp. 985-990, 2005).

The term “securin related disorder or disease associated with securin activity” refers to any disease or condition that is associated with increased or enhanced expression or activity of securin or increased or enhanced expression or activity of a gene encoding securin. Examples of such increased activity or expression can include one or more of the following: activity of the protein or expression of the gene encoding the protein is increased above the level of that in normal subjects; activity of the protein or expression of the gene encoding the protein is in an organ, tissue or cell where it is not normally detected in normal subjects (i.e. spatial distribution of the protein or expression of the gene encoding the protein is altered); activity of the protein or expression of the gene encoding the protein is increased when activity of the protein or expression of the gene encoding the protein is present in an organ, tissue or cell for a longer period than in a normal subjects (i.e., duration of activity of the protein or expression of the gene encoding the protein is increased). A normal or healthy subject is a subject not suffering from a securin related disorder or disease.

“Inhibitors,” “activators,” and “modulators” of expression or of activity are used to refer to inhibitory, activating, or modulating molecules, respectively, identified using in-vitro and in-vivo assays for expression or activity. Inhibitors of the present invention are compositions that, inhibit expression of securin or bind to, partially or totally block stimulation, decrease, prevent, delay activation, inactivate, desensitize, or down regulate the activity of securin. Samples or assays comprising securin can be treated with a composition of the present invention and compared to control samples without a composition of the present invention. Control samples (untreated with compositions of the present invention) can be assigned a relative activity value of 100%. In certain embodiments, inhibition of securin is achieved when the activity value relative to the control is about 80% or less, optionally 50% or 25, 10%, 5% or 1%.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like which would be to a degree that would prohibit administration of the compound.

A “therapeutically effective amount” or “pharmaceutically effective amount” means the amount that, when administered to a subject, produces effects for which it is administered. For example, a “therapeutically effective amount,” when administered to a subject to inhibit securin activity, is sufficient to inhibit securin activity. A “therapeutically effective amount,” when administered to a subject for treating a disease, is sufficient to effect treatment for that disease.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that are associated with the targeted or suspected disease or condition. These and other routine methods allow the clinician to select patients in need of therapy using the methods and formulations of the present invention.

The present invention provides fused heterobicyclic 2-aryl- or 2-heteroarylthiazolyl compounds of formula I as pharmaceuticals. In a preferred embodiment, fused heterobicyclic 2-aryl- or 2-heteroarylthiazolyl compounds of formula I are formulated as pharmaceuticals to treat diseases associated with increased securin activity, e.g., by inhibiting growth of cancerous cell lines, including but limited to for example, the growth of human breast carcinoma in a subject.

In general, the fused heterobicyclic 2-aryl- or 2-heteroarylthiazolyl compounds of formula I can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection). Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso AR: Remington's Pharmaceutical Sciences, 17th ed., Mack Publishing Company, Easton Pa., 1985. Suitable liquid carriers, especially for injectable solutions, include water, aqueous saline solution, aqueous dextrose solution, and glycols. In some embodiments of the present invention, the 2-arylthiazolyl compounds of Formula I suitable for use in the practice of this invention will be administered either singly or in combination with at least one other compound of this invention. The fused heterobicyclic 2-aryl- or 2-heteroarylthiazolyl compounds of formula I suitable for use in the practice of the present invention can also be administered with at least one other conventional therapeutic agent for the disease being treated. Compounds of the invention may preferably be orally administered, for example, with an inert diluent or with an assimilable edible carrier, or they may be enclosed in hard or soft shell gelatin capsules, or they may be compressed into tablets or they may be incorporated directly with the food of the diet. For oral therapeutic administration, these active compounds may be incorporated with excipients and used in the form of ingestible tablets, buccal tablets, troches, capsules, elixirs, suspensions, syrups, wafers and the like. Such compositions and preparations should contain at least 0.1% of active compound. The percentage of the compositions and preparations may, of course, be varied and may conveniently be between about 2% to about 60% of the weight of the unit. The amount of active compound in such therapeutically useful compositions is such that a suitable dosage will be obtained. Preferred compositions or preparations according to the present invention are prepared so that an oral dosage unit form contains between 10 and 1000 mg of active compound.

Aqueous suspensions of the invention can contain a fused heterobicyclic 2-aryl- or 2-heteroarylthiazolyl compound of formula I in admixture with excipients suitable for the manufacture of aqueous suspensions. Such excipients can include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions can be formulated by suspending a fused heterobicyclic 2-aryl- or 2-heteroarylthiazolyl compound of formula I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. Under ordinary conditions of storage and use, these preparations contain a preservative to prevent the growth or microorganisms.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the fused heterobicyclic 2-aryl- or 2-heteroarylthiazolyl compound of formula I in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol. The formulations of 2-arylthiazolyl compounds of formula I can be presented in unit-dose or multi-dose sealed containers, such as ampules and vials. Injection solutions and suspensions can be prepared from sterile powders, granules, and tablets of the kind previously described.

The fused heterobicyclic 2-aryl- or 2-heteroarylthiazolyl compounds of this invention can be administered orally. The amount of a compound of the present invention in the composition can vary widely depending on the type of composition, size of a unit dosage, kind of excipients, and other factors well known to those of ordinary skill in the art. In general, the final composition can comprise from, for example, 0.000001 percent by weight (% w) to 10% w of the fused heterobicyclic 2-aryl- or 2-heteroarylthiazolyl compound of formula I, preferably 0.00001% w to 1% w, with the remainder being the excipient or excipients.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient. Formulations suitable for oral administration can consist of (a) liquid solutions, such as an effective amount of the packaged nucleic acid suspended in diluents, such as water, saline or polyethylene glycol (e.g. PEG 400); (b) capsules, sachets or tablets, each containing a predetermined amount of the active ingredient, as liquids, solids, granules or gelatin; (c) suspensions in an appropriate liquid; and (d) suitable emulsions.

Pharmaceutical preparations for oral use can be obtained through combination of the compounds of the present invention with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxymethyl cellulose, hydroxypropylmethyl-cellulose or sodium carboxymethylcellulose; and gums including arabic and tragacanth; as well as proteins such as gelatin and collagen. If desired, disintegrating or solubilizing agents can be added, such as the cross-linked polyvinyl pyrrolidone, agar, alginic acid, or a salt thereof, such as sodium alginate. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The fused heterobicyclic 2-aryl- or 2-heteroarylthiazolyl compound of the present invention can also be administered in the form of suppositories for rectal administration of the drug. These formulations can be prepared by mixing the drug with a suitable non-irritating excipient, which is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The fused heterobicyclic 2-aryl- or 2-heteroarylthiazolyl compound of formula I can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention, e.g., anti-atherosclerotic medicaments.

In another embodiment, the compounds of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). In other cases, the preferred preparation can be a lyophilized powder which may contain, for example, any or all of the following: 1 mM-50 mM histidine, 0.1%-2% sucrose, 2%-7% mannitol, at a pH range of 4.5 to 5.5, that is combined with buffer prior to use.

A pharmaceutical composition of the invention can optionally contain, in addition to a fused heterobicyclic 2-aryl- or 2-heteroarylthiazolyl compound of formula I, at least one other therapeutic agent useful in the treatment of a disease or condition associated with increased securin activity. The pharmaceutical compositions are generally formulated as sterile, substantially isotonic and in full compliance with all Good Manufacturing Practice (GMP) regulations of the U.S. Food and Drug Administration. The pharmaceutical compositions of the present invention may comprise combining 2-arylthiazolyl compounds of formula I with one or more other kinase-inhibiting compounds or chemotherapeutic agents. Chemotherapeutic agents include, but are not limited to exemestane, formestane, anastrozole, letrozole, fadrozole, taxane and derivatives such as paclitaxel or docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, and Herceptin. Methods of administrating a pharmaceutical composition in accordance with the invention are not specifically restricted, and can be administered in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum. The dosage of a pharmaceutical composition according to the present invention will depend on the method of use, the age, sex, and condition of the patient.

The present invention provides methods of inhibiting securin activity in a subject for the treatment of diseases and conditions associated with increased securin activity using a fused bicyclic 2-aryl- or 2-heteroarylthiazolyl compound of formula 1. In an exemplary embodiment of the present invention, a skilled practitioner will treat a subject having a disease associated with elevated securin levels and/or activity with the compounds of the present invention.

For treatment purposes, the compositions or compounds disclosed herein can be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). The pharmaceutical formulations of the present invention can be administered, for example, one or more times daily, 3 times per week, or weekly. In an exemplary embodiment of the present invention, the pharmaceutical formulations of the present invention are orally administered once or twice daily.

In this context, a therapeutically effective dosage of the biologically active agent(s) can include repeated doses within a prolonged treatment regimen that will yield clinically significant results to alleviate one or more symptoms or detectable conditions associated with increased securin activity. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in-vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response). In alternative embodiments, an “effective amount” or “therapeutically effective dose” of the biologically active agent(s) will simply inhibit or enhance one or more selected biological activity(ies) correlated with a disease or condition, as set forth above, for either therapeutic or diagnostic purposes.

The actual dosage of biologically active agents will of course vary according to factors such as the extent of exposure and particular status of the subject (e.g., the subject's age, size, fitness, extent of symptoms, susceptibility factors, etc), time and route of administration, as well as other drugs or treatments being administered concurrently. Dosage regimens can be adjusted to provide an optimum prophylactic or therapeutic response. By “therapeutically effective dose” herein is meant a dose that produces effects for which it is administered. More specifically, a therapeutically effective dose of the compound(s) of the invention preferably alleviates symptoms, complications, or biochemical indicia of diseases associated with increased securin activity. The exact dose will depend on the purpose of the treatment, and will be ascertainable by one skilled in the art using known techniques (see, e.g., Lieberman, Pharmaceutical Dosage Forms (Vols. 1-3, 1992); Lloyd, 1999, The Art, Science, and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compound.

In an exemplary embodiment of the present invention, unit dosage forms of the compounds are prepared for standard administration regimens. In this way, the composition can be subdivided readily into smaller doses at the physicians direction. For example, unit dosages can be made up in packeted powders, vials or ampoules and preferably in capsule or tablet form. The active compound present in these unit dosage forms of the composition can be present in an amount of, for example, from about one gram to about fifteen grams or more, for single or multiple daily administration, according to the particular need of the patient. By initiating the treatment regimen with a minimal daily dose of about one gram, the blood levels of securin and the patients symptomatic relief analysis can be used to determine whether a larger or smaller dose is indicated. Effective administration of the compounds of this invention can be given at an oral dose of from, for example about 0.1 mg/kg/day to about 1,000 mg/kg/day. Preferably, administration will be from about 10/mg/kg/day to about 600 mg/kg/day, more preferably from about 25 to about 200 mg/kg/day, and even more preferably from about 50 mg/kg/day to about 100 mg/kg/day.

In certain embodiments, the present invention is directed to prodrugs of fused heterobicyclic 2-aryl- or 2-heteroarylthiazolyl compounds of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975).

After a pharmaceutical comprising a fused heterobicyclic 2-aryl- or 2-heteroarylthiazolyl compound of formula I has been formulated in a suitable carrier, it can be placed in an appropriate container and labeled for treatment of a securin related disorder, e.g., breast cancer. Additionally, another pharmaceutical comprising at least one other therapeutic agent useful in the treatment of the securin related disorder can be placed in the container as well and labeled for treatment of the indicated disease. Alternatively, a single pharmaceutical comprising a fused heterobicyclic 2-aryl- or 2-heteroarylthiazolyl compound of formula I and at least one other therapeutic agent useful in the treatment of a securin related disorder can be placed in an appropriate container and labeled for treatment. For administration of pharmaceuticals comprising a fused heterobicyclic 2-aryl- or 2-heteroarylthiazolyl compound of formula I and of pharmaceuticals comprising, in a single pharmaceutical, a fused heterobicyclic 2-aryl- or 2-heteroarylthiazolyl compound of formula I and at least one other therapeutic agent useful in the treatment of a securin related disorder, such labeling would include, for example, instructions concerning the amount, frequency and method of administration. Similarly, for administration of multiple pharmaceuticals provided in the container, such labeling would include, for example, instructions concerning the amount, frequency and method of administration of each pharmaceutical.

Based on the results of standard pharmacological test procedures described herein, the fused heterobicyclic 2-aryl- or 2-heteroarylthiazolyl compounds of formula I are useful as agents for treating, inhibiting or controlling the growth of cancerous tumor cells and associated diseases in a mammal in need thereof. The compounds of the invention are useful as agents for treating, inhibiting or controlling the growth of cancerous tumor cells and associated diseases in a mammal. In the case of cancer treatment, it is believed that many neoplasias such as leukemia, lung cancer, colon cancer, thyroid cancer, ovarian cancer, renal cancer, prostate cancer and breast cancers may be treated by effectively administering effective amounts of a fused heterobicyclic 2-aryl- or 2-heteroarylthiazolyl compound of formula I. Suitable examples of cancers for treatment using methods provided herein include carcinoma, sarcoma, lymphoma, or leukemia. The term “carcinoma” refers to a benign or malignant epithelial tumor and includes, but is not limited to, breast carcinoma, prostate carcinoma, non-small lung carcinoma, colon carcinoma, melanoma carcinoma, ovarian carcinoma, or renal carcinoma. A preferred subject or mammalian host benefiting from treatment using one or more compounds of the invention is a human.

Selected fused heterobicyclic 2-aryl- or 2-heteroarylthiazolyl compounds of formula I were tested for their activity in a securin parental cell line (HCT116 FB) and a securin knockout (KO) cell line. Certain fused bicyclic 2-aryl- or 2-heteroarylthiazolyl compounds of formula I inhibited the growth of the securin knockout cells but not securin parental cells, which were characterized by high selectivity ratios (IC₅₀ HCT116 FB/IC₅₀ securin KO), as summarized in Table I. For example, the fused heterobicyclic 2-aryl-thiazolyl compound of Example 10 exhibited a high selectivity ratio. The in-vivo activity of selected fused heterobicyclic 2-arylarylthiazolyl compounds of formula I was studied in cells growing as xenografts in athymic (nude) mice. The effect of Examples 5-7 and 10 was studied in xenografts of a human breast carcinoma cell line (MDA-MB-361).

Examples of fused heterobicyclic 2-aryl- or 2-heteroarylthiazolyl compounds of formula I were evaluated in several standard pharmacological test procedures that showed that the compounds of this invention possess significant activity in reducing the volume of tumor growth in-vivo. Based on the activity shown in the standard pharmacological test procedures, the compounds of this invention are therefore useful as anti-cancer agents. Associated cancers are selected from the group consisting of breast, colon, lung, prostate, melanoma, epidermal, leukemia, kidney, bladder, mouth, larynx, esophagus, stomach, ovary, pancreas, liver, skin and brain. In particular, the compounds of this invention possess an effect similar to HKI-272. The test procedures used and results obtained are shown below. Having described the invention, the invention is further illustrated by the following non-limiting examples.

### EXAMPLES

The syntheses of Examples 1-17 are described in Examples.

### Example 1

Preparation of 2-(4-dimethylamino-phenyl)-5-methyl-4,5,6,7-tetrahydro[1,3]thiazolo[4,5-c]pyridine-7-ol. Using the sequence of reactions illustrated below, 2-(4-dimethylamino-phenyl)-5-methyl-4,5,6,7-tetrahydro[1,3]thiazolo[4,5-c]pyridine-7-ol was prepared and mass spectral analysis was performed.

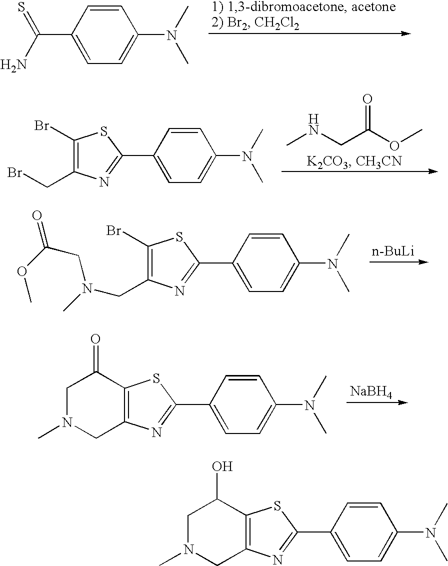

Step 1: [4-(5-bromo-4-bromomethyl-thiazol-2-yl)-phenyl]-dimethylamine. A mixture of 4-dimethylamino benzothioamide (500 mg=2.77 mmol) and 1,3-dibromoacetone (598 mg=2.77 mmol) in 20 mL of acetone was stirred at 90° C. for 4 hours. After the mixture was cooled to room temperature, 0.9 mL of 48% HBr was added. The reaction mixture was reheated to 90° C. for 1 hour. The resulting reaction mixture was concentrated, re-diluted with dichloromethane, washed with saturated NaHCO₃ aqueous solution and brine solution, dried over MgSO₄, and then evaporated to dryness. Dichloromethane (7 mL) was added to the above residue to form a solution. Bromine (213 μL=4.14 mmol) was added dropwise. After 10 hours of stirring at room temperature, the resulting reaction mixture was evaporated on a rotary evaporator, re-diluted with dichloromethane, washed with saturated NaHCO₃ aqueous solution and brine solution, dried over MgSO₄, then filtered and concentrated. The residue was chromotographed over silica, eluting with 20% of ethyl acetate in hexane to provide 320 mg of [4-(5-bromo-4-bromomethyl-thiazol-2-yl)-phenyl]-dimethyl-amine as a yellow solid; MS: m/z=374.9, 376.9, 378.9 (M+H).

Step 2: [5-bromo-2-(4-dimethylamino-phenyl)-thiazol-4-ylmethyl]-methyl-amino)-acetic acid methyl ester. A mixture of [4-(5-bromo-4-bromomethyl-thiazol-2-yl)-phenyl]-dimethyl-amine (500 mg=1.33 mmol), SAR-OMe HCl (371 mg=266 mmol) and K₂CO₃ (551 mg=3.99 mmol) in 10 mL of acetonitrile was heated to 50° C. for 10 hours. The resulting reaction mixture was diluted in ethyl acetate, washed with saturated NaHCO₃ aqueous solution and brine solution, dried over MgSO₄, then filtered and concentrated. The residue was chromatographed over silica, eluting with 30% ethyl acetate in hexane to provide 127 mg of [5-bromo-2-(4-dimethylamino-phenyl)-thiazol-4-ylmethyl]-methyl-amino)-acetic acid methyl ester as a yellow solid; MS: m/z=398.0, 400.0 (M+H).

Step 3: 2-(4-dimethylamino-phenyl)-5-methyl-5,6-didydro-4H-thiazolo[4,5-c][1,3]pyridine-7-one. Into a solution of [5-bromo-2-(4-dimethylamino-phenyl)-thiazol-4-ylmethyl]-methyl-amino)-acetic acid methyl ester (100 mg=0.25 mmol) in dry THF at −78° C., n-butyl lithium (2.5 M) in hexane (100 μL=0.25 mmol) was added dropwise. After 30 minutes of stirring, the reaction was quenched with an aqueous solution of NH₄Cl. The resulting reaction mixture was diluted with dichloromethane, washed with saturated NaHCO₃ aqueous solution and brine solution, dried over MgSO₄, then filtered and concentrated. The residue was chromatographed over 40 g of silica in a column, eluting with 30% of hexane in ethyl acetate to provide 10.0 mg of 2-(4-dimethylamino-phenyl)-5-methyl-5,6-didydro-4H-thiazolo[4,5-c]pyridine-7-one as a yellow solid; MS: m/z=288.1 (M+H).

Step 4: 2-(4-dimethylamino-phenyl)-5-methyl-4,5,6,7-tetrahydro[1,3]thiazolo[4,5-c]pyridine-7-ol. Into a solution of 2-(4-dimethylamino-phenyl)-5-methyl-5,6-didydro-4H-thiazolo[4,5-c][1,3]pyridine-7-one (50 mg=0.17 mmol) in 5 mL of THF and 5 mL of methanol, sodium borohydride (6.6 mg=0.17 mmol) was added in portion. After 2 hours stirring at room temperature, the resulting reaction mixture was quenched with water, diluted with ethyl acetate, washed with saturated NaHCO₃ aqueous solution and brine solution, dried over MgSO₄, then filtered and concentrated. The residue was chromatographed over silica, eluting with 30% hexane in ethyl acetate to provide 35 mg of 2-(4-dimethylamino-phenyl)-5-methyl-4,5,6,7-tetrahydro[1,3]thiazolo[4,5-c]pyridine-7-ol as a yellow solid.

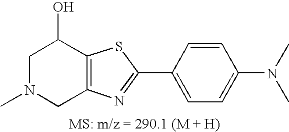

### Example 2

Preparation of 2-(4-dimethylamino-phenyl)-4,5,6-tetrahydro[1,3]thiazolo[4,5-c]pyridin-7-ol. Using the sequence of reactions illustrated in Method A and Method B, a racemic mixture of 2-(4-dimethylamino-phenyl)-4,5,6,7-tetrahydro[1,3]thiazolo[4,5-c]pyridin-7-ol was prepared and mass spectral analysis was performed.

Method A: 2-(4-dimethylamino-phenyl)-4,5,6,7-tetrahydro[1,3]thiazolo[4,5-c]pyridin-7-ol was synthesized analogously to the compound of Example 1, except that Steps 4 and 7 were modified as indicated below.

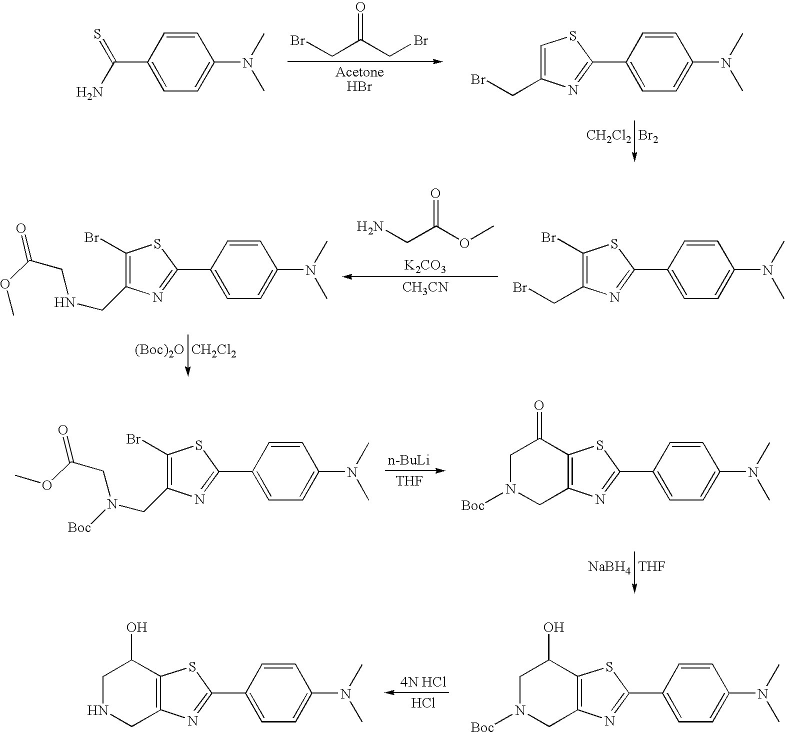

Step 4: Into a solution of the intermediate compound (500 mg=1.3 mmol) in 20 mL of dichloromethane, (t-Boc)₂O (426 mg=1.95 mmol) was added. The reaction mixture was stirred at room temperature overnight. The resulting reaction mixture was diluted with dichloromethane, washed with saturated NaHCO₃ aqueous solution and saturated NaCl aqueous solution, dried over MgSO₄, filtered and concentrated. The residue was chromatographed on a column using 40 grams of silica, eluting with 20% ethyl acetate in hexane to provide 600 mg of the t-Boc protected compound as colorless oil. MS: m/z=484.1, 486.0.

Step 7: Into a solution of the t-Boc protected compound (826 mg=2.2 mmol) in 50 ml of dichloromethane, 2N HCl in diethyl ether (5.39 mL=10.8 mmol) was added dropwise. The reaction mixture was stirred at room temperature overnight, then evaporated. The residue remaining was diluted with dichloromethane, washed with saturated NaHCO₃ aqueous and saturated NaCl aqueous solutions, dried over MgSO₄, filtered, and concentrated. The residue was chromatographed using a column with 40 g of silica, eluting with 10% methanol in dichloromethane to provide 390 mg of product as a white solid. MS: m/z=276.1 (M+H).

Method B:

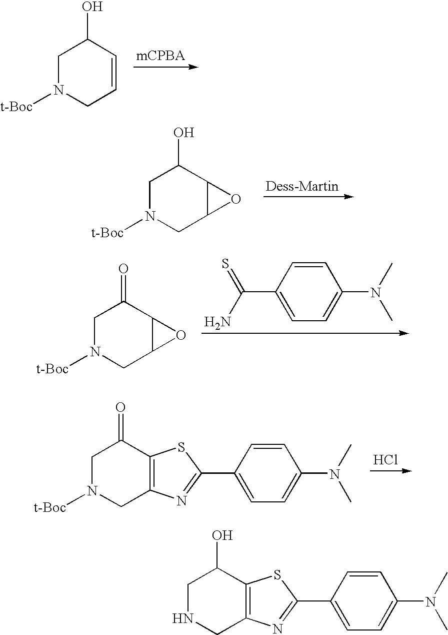

Step 1: 5-dydroxy-7-oxa-3-aza-bicyclo[4.1.0]heptane-3-carbonxylic acid tert-butyl ester. Into a solution of 3-hydroxy-3,6-dihydro-2H-pyridine-1-carboxylic acid tert-butyl eter (500 mg=2.5 mmol) in 10 mL of dichloromethane, 77% mCPBA (1.12 g=5 mmol) was added in portion. After 12 hours of stirring at room temperature, the reaction mixture was diluted with dichloromethane, washed with saturated NaHCO₃ aqueous solution and brine solution, dried over MgSO₄, filtered over a magnesol bed, concentrated and dried further in vacuo to provide 506 mg of 5-dydroxy-7-oxa-3-aza-bicyclo[4.1.0]heptane-3-carbonxylic acid tert-butyl ester as a white solid.

Step 2: 5-oxo-7-oxa-3-aza-bicyclo[4.1.0]heptane-3-carboxylic acid tert-butyl ester. Into a solution of 5-dydroxy-7-oxa-3-aza-bicyclo[4.1.0]heptane-3-carboxylic acid tert-butyl ester (250 mg=1.25 mmol) in 5 mL of dichloromethane, Dess-Martin reagent (650 mg=1.5 mmol) was added in portion. After 12 hours of stirring at room temperature, the reaction solvent was evaporated. The residue was diluted with ethyl ether, then stirred and filtered. The filtrate was washed with saturated NaHCO₃ aqueous solution and brine solution, filtered through a magnesol bed, concentrated and dried further in vacuo to obtain 230 mg of 5-oxo-7-oxa-3-aza-bicyclo[4.1.0]heptane-3-carboxylic acid tert-butyl ester as a white semi-solid.

Step 3: 2-(4-dimethylamino-phenyl)-7-hydroxy-6,7-dihydro-4H-thiazolo[4,5-c][1,3]pyridine-5-carboxylic acid tert-butyl ester. A mixture of 5-oxo-7-oxa-3-aza-bicyclo[4.1.0]heptane-3-carboxylic acid tert-butyl ester (2.0 g=9.37 mmol) and 4-dimethylamino-thiobenzamide (1.93 g=9.37 mmol) in 50 ml of ethanol was heated to 60° C. for 16 hours. The resulting reaction mixture was then concentrated. The residue was chromatographed over silica, eluting with 30% ethyl acetate in hexane to provide 1.57 g of 2-(4-dimethylamino-phenyl)-7-hydroxy-6,7-dihydro-4H-thiazolo[4,5-c]pyridine-5-carboxylic acid tert-butyl ester as a light yellow solid; MS: m/z=376.1 (M+H).

Step 4: 2-(4-dimethylamino-phenyl)-4,5,6,7-tetrahydro[1,3]thiazolo[4,5-c]pyridin-7-ol. Into a solution of 2-(4-dimethylamino-phenyl)-7-hydroxy-6,7-dihydro-4H-thiazolo[4,5-c][1,3]pyridine-5-carboxylic acid tert-butyl ester (1.57 g=4.18 mmol) in 40 mL of dichloromethane, 4N HCl in 1,4-dioxane (6.27 ml=25 mmol) was added dropwise. After 12 hours of stirring, the reaction mixture was evaporated, treated with Na₂CO₃ aqueous solution (2.7 g in 200 ml of water), extracted with chloroform, washed with brine solution, dried over MgSO₄, then filtered and concentrated. The residue was chromatographed over silica (eluting with 20% of methanol in dichloromethane) to provide 904 mg of 2-(4-dimethylamino-phenyl)-4,5,6,7-tetrahydro[1,3]thiazolo[4,5-c]pyridin-7-ol as a light yellow solid. MS: m/z=276.1 (M+H).

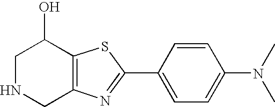

2-(4-dimethylamino-phenyl)-4,5,6-tetrahydro-thiazolo[4,5-c]pyridin-7-ol, MS: m/z=276.1 (M+H)

### Examples 3 and 4

As illustrated below, the racemic mixture was separated and mass spectral analysis was performed on each compound.

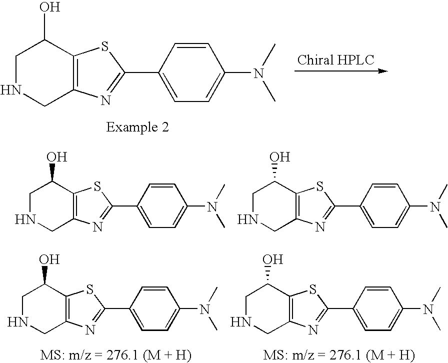

### Example 5

Preparation of (7R,7S)-2-(4-pyrrolidin-1-yl-phenyl)-4,5,6,7-tetrahydro[1,3]thiazolo[4,5-c]pyridin-7-ol. Using the sequence of reactions illustrated below, a racemic mixture of (7R,7S)-2-(4-pyrrolidin-1-yl-phenyl)-4,5,6,7-tetrahydro[1,3]thiazolo[4,5-c]pyridin-7-ol was prepared and mass spectral analysis was performed. The product was synthesized analogously as described above, except for step 1, the preparation of 4-pyrrolidinylbenzonitrile

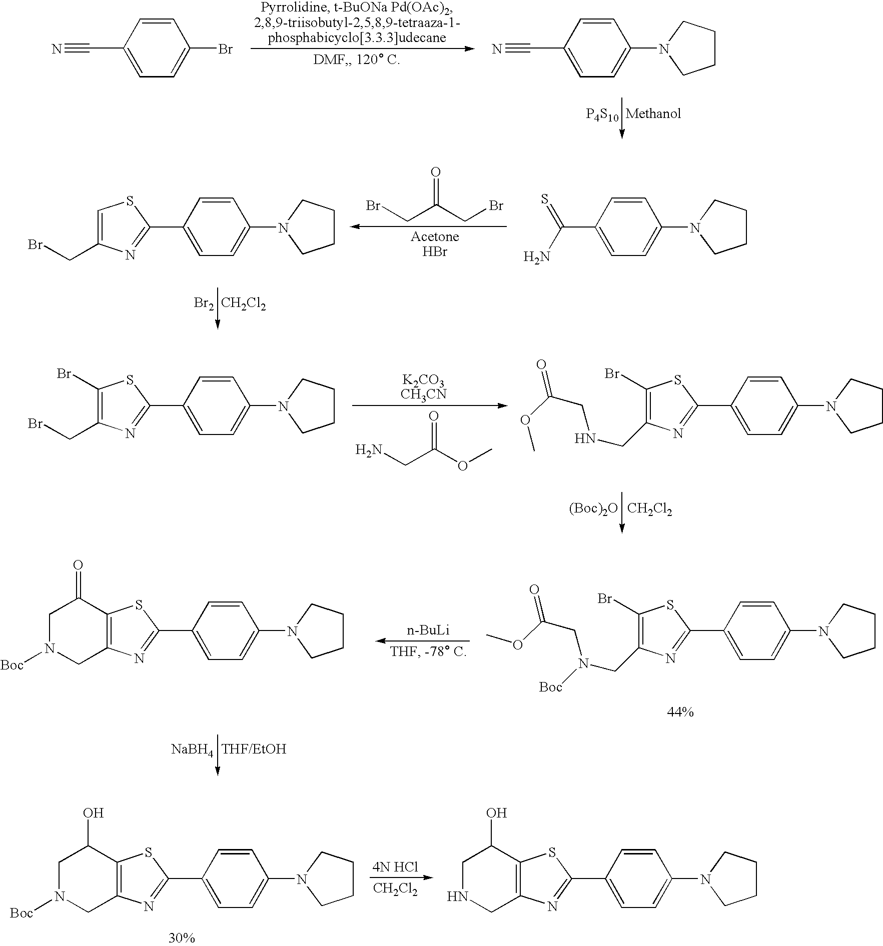

### Method A

Step 1: Synthesis of 4-pyrrolidinylbenzonitrile. A solution mixture of 4-bromobenzonitrile (2.0 g=11 mmol), pyrrolidine (1.84 ml=22 mmol), t-BuONa (1.6 g=16.5 mmol), Pd(Oac)₂ (99 mg=0.44 mmol) and 2,8,9-triisobutyl-2,5,8,9-tetraaza-1-phosphabicyclo[3.3.3]udecane (313 μL=0.88 mmol) in 20 mL of DMF was stirred at 120° C. for 2 hours. The resulting reaction mixture was diluted with ethyl acetate, washed with water and saturated NaCl aqueous solution, dried over MgSO₄, filtered and concentrated. The residue was chromatographed in a column over 120 g of silica, eluting with 10% ethyl acetate in hexane to provide 1.5 g of 4-pyrrolidinylbenzonitrile as a light yellow solid. MS: m/z=173.2 (M+H).

The product was also synthesized analogously to a procedure described above for Example 2 (Method B).

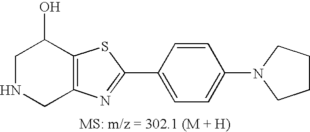

### Examples 6 and 7

As illustrated below, the racemic mixture was separated and mass spectral analysis was performed on each compound.

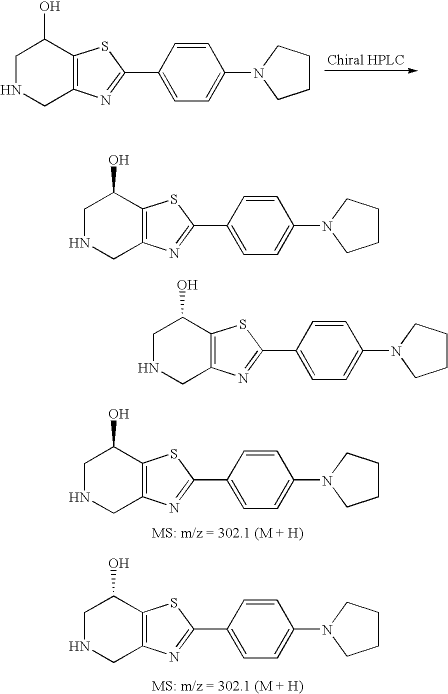

### Example 8

Using the sequence of reactions illustrated below, 2-(2-hydroxymethyl-4-dimethylamino-phenyl)-4,5,6,7-tetrahydro[1,3]thiazolo[4,5-c]pyridine-7-ol was prepared and mass spectral analysis was performed.

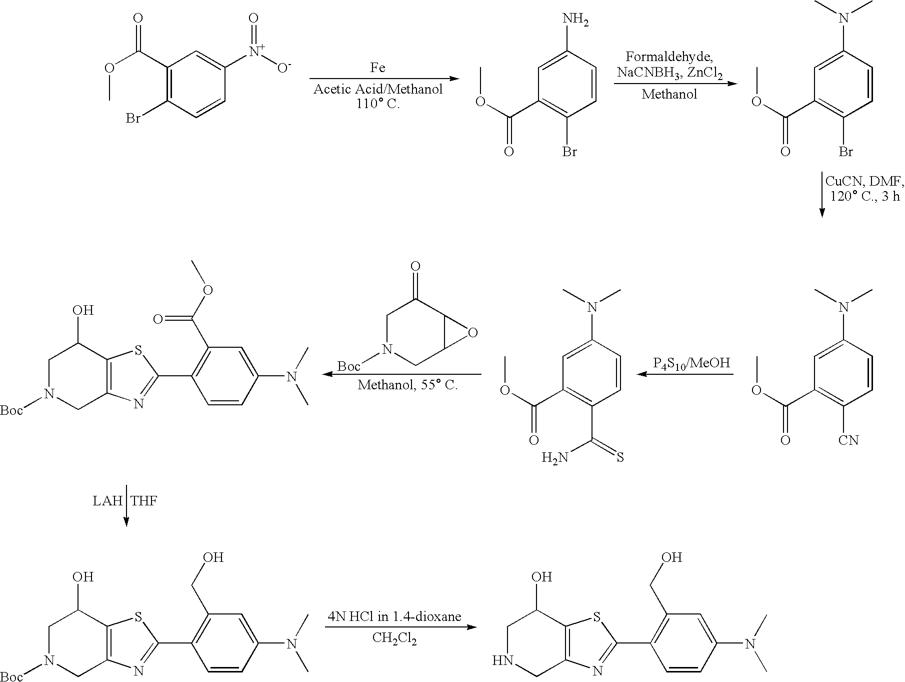

Step 1: Into a solution mixture of methyl-2-bromo-5-nitrobenzoate (25 g=96.1 mmol) and Iron (10.77 g=192.28 mmol) in 100 mL of methanol, 94 ml of acetic acid was added. The reaction mixture was heated to 80° C. for 3 hours. The precipitate was suction filtered. The filtrate was evaporated. The remaining residue was re-diluted with ethyl acetate, washed with Na₂CO₃ aqueous solution and saturated NaCl aqueous solution, dried over MgSO₄, filtered and concentrated. The crude residue was chromatographed in a column using 120 g of silica, eluting with 20% ethyl acetate in hexane to provide 20.1 g of the aniline compound as light yellow oil. MS: m/z=230.0, 232.0 (M+H).

Step 2: Into a mixture of the aniline compound (10 g=43.47 mmol), 37% formaldehyde (19 mL=195.65 mmol), and sodium cyanoborohydride (8.19 g=130.41 mmol) in 150 mL of methanol at 0-5° C., Zinchloride (2.96 g=21.73 mmol) was added in portion. The reaction mixture was stirred at room temperature for 14 hours. The reaction solvent was evaporated. The remaining residue was re-diluted with ethyl acetate, washed with saturated NaHCO₃ aqueous solution and saturated NaCl aqueous solution, dried over MgSO₄, then filtered, concentrated and dried further in-vacuo to provide 13.57 g of the N,N-dimethylaniline compound as a yellow solid. MS: m/z=258.0, 260.0 (M+H).

Step 3: A solution mixture of the dimethylaniline compound (6.87 g=22 mmol) and copper (I) cyanide (6.31 g=44 mmol) in 50 mL of DMF was stirred at 110° C. for 1 hour, then poured into cold ethyl acetate. The precipitate was filtered. The filtrate was washed with saturated NaHCO₃ aqueous solution and saturated NaCl aqueous solution, dried over MgSO₄, then filtered and concentrated. The crude residue was chromatographed over a 120 g of silica column, eluting with 40% ethyl acetate in hexane to provide 2.7 g of the cyano compound as a white solid. MS: m/z=205.1 (M+H).

Step 4: Into a solution mixture of the cyano compound (2.3 g=11.27 mmol) in 20 ml of methanol at 0° C., P₄S₁₀ (5.0 g=11.27 mmol) was added in portion. The reaction mixture was allowed to warm to room temperature over night. The resulting reaction mixture was suction filtered, washed with 50% of dichloromethane in hexane and dried further in-vacuo to provide 2.6 g of the thioacetamide as a white solid. MS: m/z=239.1 (M═H).

Step 5: A mixture of the thioacetamide (300 mg=1.4 mmol) and 5-oxo-7-oxa-3-aza-bicyclo[4.1.0]heptane-3-carboxylic acid tert-butyl ester (298.5 mg=1.4 mmol) in 4 mL of methanol was stirred at 60° C. for 3 hours. The resulting reaction mixture was concentrated. The residue was chromatographed over a 40 g silica column, eluting with 20% acetonitrile in dichloromethane to provide 146 mg of the protected thiazole as a yellow semi solid. MS: m/z=434.2 (M+H).

Step 6: Into a solution of the protected thiazole (600 mg=1.75 mmol) in 5 ml of THF at 0° C., LAH (133 mg=3.5 mmol) was added in portion. The reaction was stirred for 0.5 h then quenched with water. The resulting mixture was diluted with ethyl acetate, washed with water and saturated NaCl aqueous solution, dried over MgSO4, filtered and concentrated. The residue was chromatographed over a 40 g silica column, eluting with 20% of acetonitrile in dichloromethane to provide 244 mg of the thiazole as a light yellow solid. MS: m/z=406.2 (M+H).

Step 7: Into a solution of the thiazole (244 mg=0.6 mmol) in 10 mL of dichloromethane, 4N HCl in 1,4-dioxane (602 μL=2.48 mmol) was dropwise added. The reaction mixture was stirred at room temperature for 14 h, and then concentrated. The residue was re-diluted with chloroform, washed with Na₂CO₃ aqueous solution and saturated NaCl solution, dried over MgSO₄, filtered and concentrated. The crude residue was chromatographed over a 40 g silica column, eluting with 20% of methanol in dichloromethane to provide 111.1 mg of the product 2-(2-hydroxymethyl-4-dimethylamino-phenyl)-4,5,6,7-tetrahydro[1,3]thiazolo[4,5-c]pyridine-7-ol, as a light yellow solid. MS: m/z=306.1 (M+H).

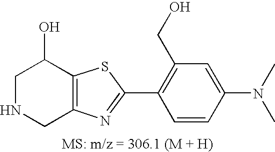

### Example 9

Preparation of methyl 5-(dimethylamino)-2-(7-hydroxy-4,5,6,7-tetrahydro[1,3]thiazolo[4,5-c]pyridin-2-yl)benzoate. Example 9 was synthesized analogously to Step 7 of Example 8.

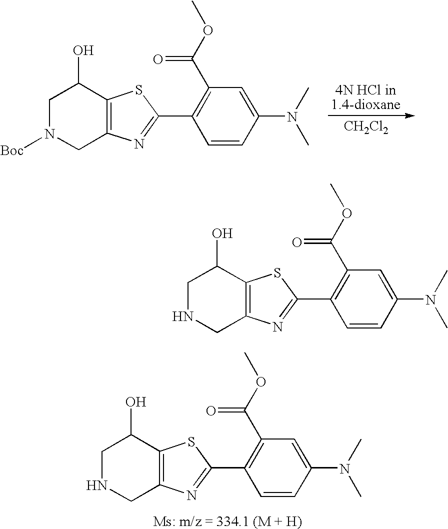

### Examples 10-12

Preparation of (7R,7S)-2-(2-hydroxymethyl-4-pyrrolidin-1-yl-phenyl)-4,5,6,7-tetrahydro[1,3]thiazolo[4,5-c]pyridin-7-ol. Example 10 was synthesized analogously to Example 9, except for Step 2.

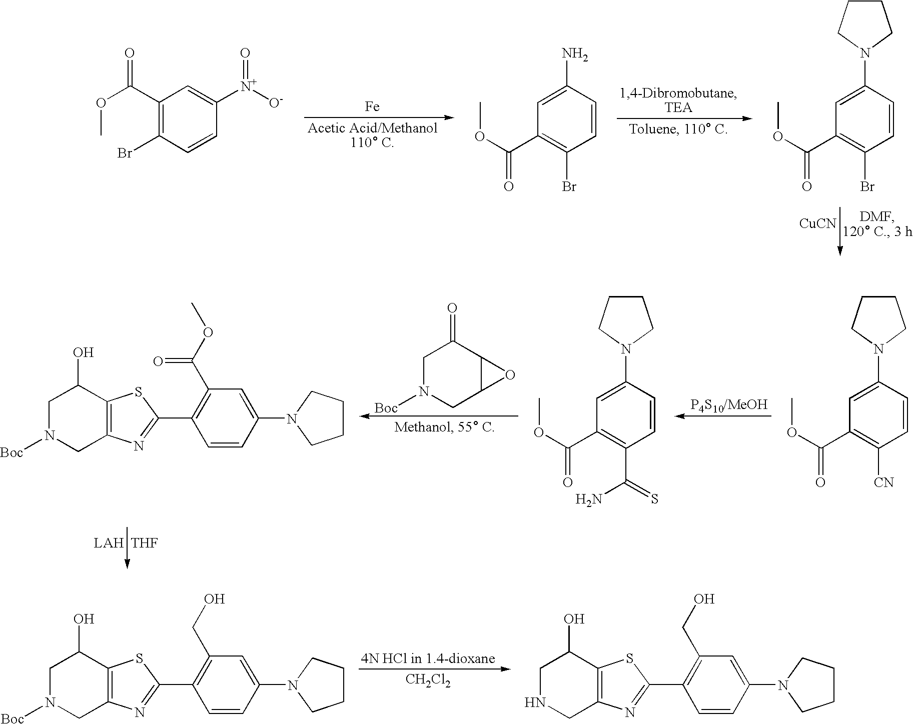

Step 2: Into a solution of the aniline compound (10 g=43.48 mmol) and triethylamine (TEA. 11.53 mL=86.96 mmol) in 100 mL of toluene, 1,4-dibromobutane (10.39 mL=86.96 mmol) was added. The reaction mixture was heated to 100° C. for 5 h. The resulting reaction mixture was concentrated, re-diluted with ethyl acetate, washed with saturated NaHCO₃ aqueous solution and NaCl aqueous solution, dried over MgSO₄, then filtered and concentrated. The residue was chromatographed over a 120 g silica column, eluting with 20% ethyl acetate in hexane to provide 7.1 g of the product 2-(4-pyrrolidin-1-yl-2-methylhydroxy-phenyl)-4,5,6-tetrahydro-thiazolo[4,5-c]pyridin-7-ol as a light yellow solid, MS: m/z 284.1, 286.1 (M+H).

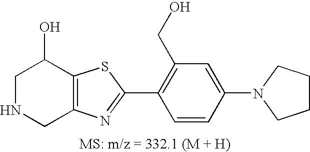

The racemic mixture was separated and mass spectral analysis was performed on each compound.

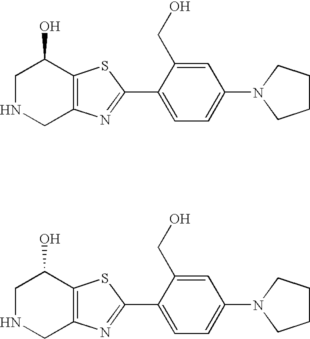

### Examples 13-15

Methyl 5-(4-pyrrolidin-1-yl)-2-(7-hydroxy-4,5,6,7-tetrahydro[1,3]thiazolo[4,5-c]pyridin-2-yl)benzoate is synthesized analogous to Step 7 of Example 10.

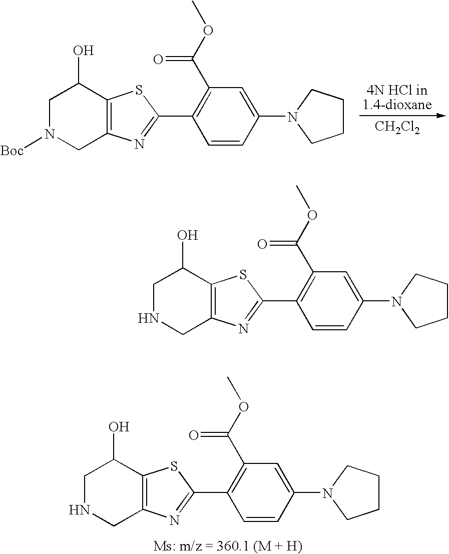

In an analogous manner, 2-(2-amino-4-pyrrolidin-1-yl-phenyl)-4,5,6,7-tetrahydro[1,3]thiazolo[4,5-c]pyridin-7-ol, MS: m/z=419.3 (M+H) and 2-(2-nitro-4-pyrrolidin-1-yl-phenyl)-4,5,6,7-tetrahydro[1,3]thiazolo[4,5-c]pyridin-7-ol, MS: m/z=316.4 (M+H) were prepared and characterized.

### Examples 16 and 17

Using the sequence of reactions illustrated below, 2-[3-bromo-4-(dimethylamino)phenyl]-6,7-dihydro-4H-pyrano[3,4-d][1,3]thiazol-7-ol and 2-[4-(dimethylamino)phenyl]-6,7-dihydro-4H-pyrano[3,4-d][1,3]thiazol-7-ol were prepared and mass spectral analysis was performed on each compound.

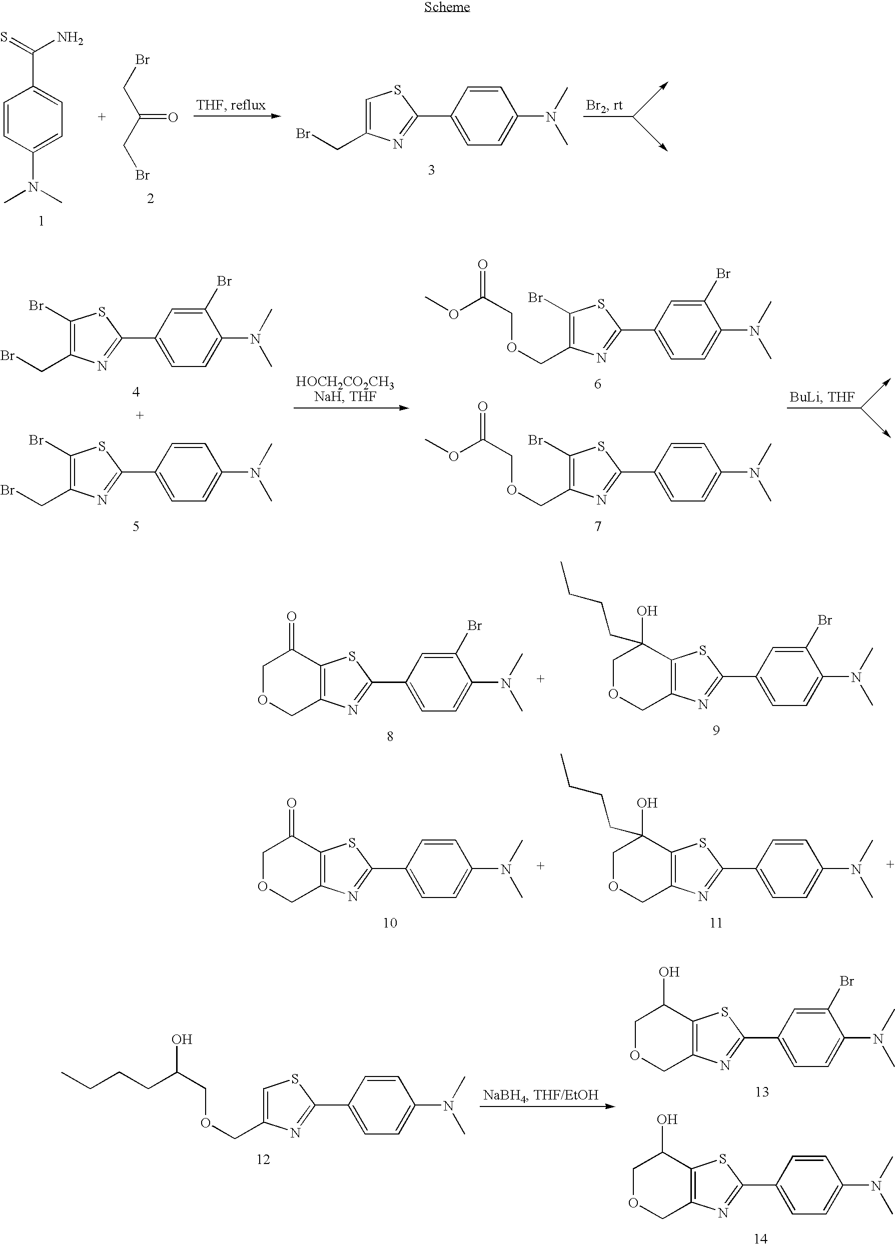

Step A. Preparation of {4-[5-bromo-4-(bromomethyl)-1,3-thiazol-2-yl]phenyl}dimethylamine and 2-bromo-4-[5-bromo-4-(bromomethyl)-1,3-thiazol-2-yl]phenyl}dimethylamine. 4-Dimethylaminothiobenzamide (760 mg, 4.22 mmol) and 1,3-dibromoacetone (1.216 g, 4.22 mmol) in anhydrous THF (30 ml) was refluxed for 5 hours. The solution was cooled to room temperature. The residue was used in next step without further purification due to the instability of bromo compounds. Bromine (0.33 ml, 6.33 mmol) was added. The reaction mixture was stirred at room temperature for 17 hours, additional bromine (0.11 ml, 2.11 mmol) was added and stirred for an additional 4 hours, then quenched with saturated NaHCO₃ solution, and then extracted with CH₂Cl₂ several times. The combined organic layers were washed with brine, dried over Na₂SO₄, then filtered and concentrated. The residue was separated by a silica gel column (CH₂Cl₂-HCN=40:1) to give {4-[5-bromo-4-(bromomethyl)-1,3-thiazol-2-yl]phenyl}dimethylamine, MS: m/z 377 (M+H) and 2-bromo-4-[5-bromo-4-(bromomethyl)-1,3-thiazol-2-yl]phenyl}dimethylamine, MS: m/z 456 (M+H).

Step B. Preparation of methyl ({5-bromo-2-[3-bromo-4-(dimethylamino)phenyl]-1,3-thiazol-4-yl}methoxy)acetate and methyl({5-bromo-2-[4-(dimethylamino)phenyl]-1,3-thiazol-4-yl}methoxy)acetate. Into a mixture of {4-[5-bromo-4-(bromomethyl)-1,3-thiazol-2-yl]phenyl}dimethylamine and 2-bromo-4-[5-bromo-4-(bromomethyl)-1,3-thiazol-2-yl]phenyl}dimethylamine prepared above, methyl glyconate (0.65 ml, 8.44 mmol) in anhydrous THF (30 ml) was added NaH (338 mg, 8.44 mmol). The reaction mixture was stirred at room temperature overnight, quenched with H₂O, and extracted with CH₂Cl₂ several times. The combined organic layers were washed with brine, dried (Na₂SO₄), filtered and concentrated. The residue was separated by flash chromatography (hexane/EtOAc=2:1) to give 514 mg of methyl({5-bromo-2-[3-bromo-4-(dimethylamino)phenyl]-1,3-thiazol-4-yl}methoxy)acetate as a yellow solid, MS: m/z 465 (M+H) and 443 mg of methyl({5-bromo-2-[4-(dimethylamino)phenyl]-1,3-thiazol-4-yl}methoxy)acetate as a brown solid, MS: m/z 386 (M+H).

Step C. Preparation of 2-(4-dimethylamino-phenyl)-4H-pyrano[3,4-d]thiazol-7-one and 7-butyl-2-[4-(dimethylamino)phenyl]-6,7-dihydro-4H-pyrano[3,4-d][1,3]thiazol-7-ol. Into a solution of methyl({5-bromo-2-[4-(dimethylamino)phenyl]-1,3-thiazol-4-yl}methoxy)acetate (144 mg, 0.374 mmol) in anhydrous THF (6 mL) cooled in an dry ice-ether bath (−87° C.) was added a solution of BuLi in hexane (1.6 M, 0.26 mL, 0.411 mmol). The reaction mixture was stirred at −87° C. for 4 h, quenched with saturated NH₄Cl. After warming to room temperature, the organic layer was separated and the aqueous layer was extracted with CH₂Cl₂ three times. The combined organic layers were dried over Na₂SO₄, filtered and concentrated. The residue was separated by a silica gel column (hexane/EtOAc=2:1) to give 20 mg of 2-(4-dimethylamino-phenyl)-4H-pyrano[3,4-d]thiazol-7-one, MS: m/z 275 (M+H) and 32 mg of 7-butyl-2-[4-(dimethylamino)phenyl]-6,7-dihydro-4H-pyrano[3,4-d][1,3]thiazol-7-ol, MS: m/z 333 (M+H) as well as 13 mg of 1-({2-[4-(dimethylamino)phenyl]-1,3-thiazol-4-yl}methoxy)hexan-2-ol, MS: m/z 335 (M+H). 2-(3-Bromo-4-dimethylamino-phenyl)-4H-pyrano[3,4-d]thiazol-7-one and 2-[3-bromo-4-(dimethylamino)phenyl]-7-butyl-6,7-dihydro-4H-pyrano[3,4-d][1,3]thiazol-7-ol were prepared by following the same procedure. 2-(3-Bromo-4-dimethylamino-phenyl)-4H-pyrano[3,4-d]thiazol-7-one, MS: m/z 354 (M+H); 2-[3-Bromo-4-(dimethylamino)phenyl]-7-butyl-6,7-dihydro-4H-pyrano[3,4-d][1,3]thiazol-7-ol, MS: m/z 412 (M+H).

Preparation of 2-[3-bromo-4-(dimethylamino)phenyl]-6,7-dihydro-4H-pyrano[3,4-d][1,3]thiazol-7-ol and 2-[4-(dimethylamino)phenyl]-6,7-dihydro-4H-pyrano[3,4-d][1,3]thiazol-7-ol. Into a solution of 2-(3-bromo-4-dimethylamino-phenyl)-4H-pyrano[3,4-d]thiazol-7-one (71 mg, 0.2 mmol) in a 1:1 mixture of THF/C₂H₅OH (3 mL) was added NaBH₄ (15 mg, 0.4 mmol). The reaction mixture was stirred at room temperature for 5 hours and then concentrated. The residue was separated by a silica gel column (hexane/EtOAc=2:1) to give 18 mg of 2-[3-bromo-4-(dimethylamino)phenyl]-6,7-dihydro-4H-pyrano[3,4-d][1,3]thiazol-7-ol, MS: m/z 356 (M+H). Following the same procedure, 2-[4-(dimethylamino)phenyl]-6,7-dihydro-4H-pyrano[3,4-d][1,3]thiazol-7-ol was prepared, MS: m/z 277 (M+H).

### Pharmacological Testing

IC₅₀ analysis was performed for selected fused heterobicyclic 2-aryl or 2-heteroarylthiazolyl compounds that were tested on HCT116 cells and Securin “knock out” cell lines. A securin screen was performed on selected fused heterobicyclic 2-aryl- or 2-heteroarylthiazolyl compounds to find compounds that preferentially inhibited the growth of the securin knockout cells, but did not inhibit the growth of securin parental cells (HCT116 FB).

Cell Culture—HCT116 and securin “knock out” cells. HCT116 and securin “knock out” cells (D8 and F3) were grown in RPMI+5% fetal bovine serum and gentamycin. They were maintained in a humidified 37° C. incubator with 5% CO₂. Since the securin “knock out” cells have active chromosomal instability, cells were used at a low passage number of 2-13 passages. At or before passage 13 a new vial of cells was thawed and used for the screen. Cell seeding—HCT116 cells and securin “knock out” cells were trypsinized and suspenended in RPMI+5% FBS at a density of 27,000 cells/mL. HCT116 and securin “knock out” cells were seeded separately in wells of a 96-well plate (Falcon Cat # 35-3872) at a cell density of 4000 cells per well. Cells were seeded in 150 μL of RPMI media containing 5% FBS and gentamycin. Cell seeding was performed under aseptic conditions. Cells were allowed to attach and grow overnight in a humidified 37° C. incubator.

Compound Dilution and Dispensation. A series of stock solutions, 10 mg/ml of 2-aryl- or 2-heteroarylthiazolyl compounds to be tested, were brought to room temperature. The compounds were diluted 1:50 in RPMI+5% FBS (Highest concentration 200 μg/mL). The compounds were then serially diluted 1:3 across a deep-well titration plate resulting in final concentrations in the plate of 200, 66.7, 22.2, 7.4, 2.4, 0.8 0.27, 0.091, 0.03, 0.01, and 0.0034 μg/mL with one well left without a compound. 50 μL of this dilution series was added to the 150 μL of cells to result in a final concentration of compound in with the cells of 50, 16.6, 5.5, 1.8, 0.6, 0.2, 0.068, 0.023, 0.0076, 0.0025, and 0.00084 μg/mL. The dilution and addition of compounds to the plates is performed using a Multimek 96 Robot (Beckman Coulter).

Incubation and analysis. After dosing, the cells in the 96 well plates were returned to the humidified 37° C. incubator. The compounds remained on the cells for 5 days. After 5 days, the cells were fixed to the plates with the addition of 50 μL of 50% (vol/vol) Trichloro Acetic Acid (1% final concentration). The cells were fixed at 4° C. for 1 hour. The plates were washed 5 times in distilled water and allowed to dry. The cells were stained with 0.4% sulforhodamine B (Sigma) in 1% acetic acid. The cells were stained for 15 minutes at room temperature. The plates were then washed 3 times in 1% acetic acid. The plates were dried and the dye was solubilized in 10 mM Tris at pH 8.0. The plates were read on the Absorbence 560 setting on a Victor² V Model 1420 Multilabel HTS counter (Perkin Elmer).

Analysis of Results—IC₅₀ data. Absorbence data was read off of the Victor and was converted to a percentage of the control (untreated) values. IC₅₀ values were graphed and the point at which a 50% inhibition of the control level was determined. A hyperbolic model that forces a curve between 0% inhibition and 100% inhibition was used. If the model was not able to determine an IC₅₀, the IC₅₀ was assigned to the lowest concentration that gives a 50% or greater decrease in absorbence compared to control levels. If the calculated IC₅₀ value for a cell line exceeds the top concentration for the assay (typically 50 μg/mL), the assay was scored as >50.

Selectivity Ratio. The Selectivity Ratio is the ratio of IC₅₀s between the parent cell line and the knock out cell line and is always a positive number and is typically an integer.

$$Selectivity Ratio = \frac{{IC}_{50} HCT 116 FB ( µg \text{/} mL )}{{IC}_{50} Securin knockout ( µg \text{/} mL )}$$

IC₅₀ data and selectivity ratios of selected fused heterobicyclic 2-arylthiazolyl compounds are summarized in Table 1.

TABLE 1
IC₅₀ data and selectivity ratios of
selected 2-arylthiazolyl compounds

| | IC₅₀ (μM) | IC₅₀ (μM) | IC₅₀ (μM) | Selectivity | |
|---|---|---|---|---|---|
| | Securin^{+/+} | Securin^{-/-} | Securin^{-/-} | Ratio | |
| Example | HCT116 | D8 | F3 | D8 | F3 |
| 1 | 23.9 | 2.48 | 3.07 | 10 | 8 |
| 2 | 86.4 | 0.931 | 1.28 | 93 | 68 |
| 3 | 66.4 | 0.395 | 0.555 | 168 | 120 |
| 4 | 169 | 2.19 | 3.39 | 77 | 50 |
| 5 | 22.8 | 0.680 | 0.930 | 34 | 25 |
| 6 | 14.5 | 0.450 | 0.429 | 32 | 34 |
| 7 | 36.4 | 21.6 | 9.67 | 2 | 4 |
| 8 | 100 | 0.0989 | 0.107 | 1011 | 936 |
| 9 | 100 | 0.530 | 0.670 | 189 | 149 |
| 10 | 80.6 | 0.0808 | 0.0944 | 998 | 854 |
| 13 | 59.2 | 0.342 | 0.516 | 173 | 115 |
| 11 | 91.4 | 0.0406 | 0.0483 | 2251 | 1892 |
| 12 | 96.2 | 0.553 | 1.04 | 174 | 92 |
| 14 | 48.1 | 0.0379 | 0.0486 | 1269 | 990 |
| 15 | 28.4 | 2.95 | 4.60 | 10 | 6 |
| 16 | 41.6 | 14.3 | 15.2 | 3 | 3 |
| 17 | 51.1 | 1.12 | 1.53 | 46 | 33 |

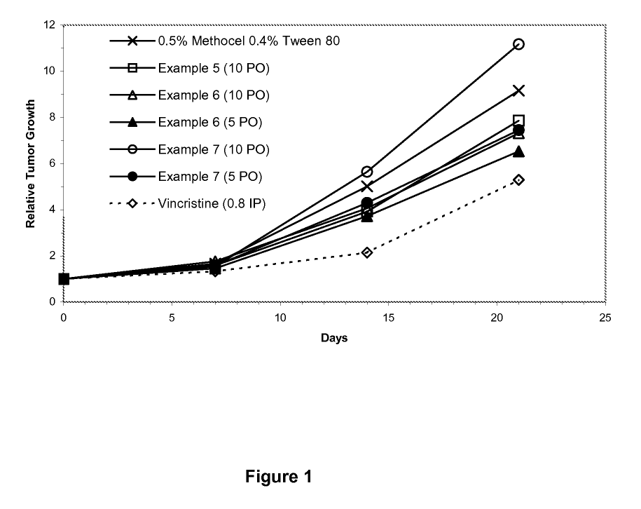

In-vivo Activity in Tumor Xenografts. The effectiveness of Examples 5-7 versus Vincristine at controlling tumor growth was studied and the results are summarized in Table 2 and FIG. 1. The 2-arylthiazolyl compounds were were formulated 0.5% Methocel™ (methylcellulose) and 0.4% Tween 80™ (polysorbate-80 and oleic acid) and administered daily orally (PO), by gavage or intraperiotenially (IP) by syringe. Tumor mass [(length×width²)/2] was determined every 7 days. Inhibition of tumor growth was calculated relative to conventional (vehicle treated) controls.

TABLE 2
Effectiveness of Selected 2-Arylthiazolyl Compounds versus Vincristine in Xenografts
of a Human Breast Carcinoma Cell Line (MDA-MB-361).

| a | | c | | b | c | | b | | | |
|---|---|---|---|---|---|---|---|---|---|---|
| Drug Treatment | b | % | d | Day | % | d | Day | c | d | e |
| mg/kg/dose | Day 7 | T/C | (p) | 14 | T/C | (p) | 21 | % T/C | (p) | S/T |
| 0.5% Methocel | 1.66 | | | 5.01 | | | 9.16 | | | 15/15 |
| 0.4% Tween 80 | | | | | | | | | | |
| Example 5 (10 PO) | 1.59 | 96 | 0.29 | 3.92 | 78 | 0.30 | 7.86 | 86 | 0.70 | 10/10 |
| Example 6 (10 PO) | 1.76 | 106 | 0.80 | 4.06 | 81 | 0.35 | 7.32 | 80 | 0.61 | 10/10 |
| Example 6 (5 PO) | 1.46 | 88 | 0.07 | 3.72 | 74 | 0.17 | 6.53 | 71 | 0.16 | 9/10 |
| Example 7 (10 PO) | 1.54 | 93 | 0.18 | 5.64 | 113 | 0.51 | 11.17 | 122 | 0.79 | 10/10 |
| Example 7 (5 PO) | 1.57 | 95 | 0.24 | 4.30 | 86 | 0.33 | 7.45 | 81 | 0.63 | 10/10 |
| 2-(4-pyrrolidiny-1- | 1.57 | 95 | 0.30 | 3.93 | 78 | 0.21 | 8.73 | 95 | 0.80 | 10/10 |
| ylphenyl)-4,5,6,7- | | | | | | | | | | |
| tetrahydro-1,3- | | | | | | | | | | |
| benzothiazol-7-ol (50 | | | | | | | | | | |
| PO) | | | | | | | | | | |
| 2-(4-pyrrolidiny-1- | 1.67 | 101 | 0.61 | 5.48 | 109 | 0.65 | 12.07 | 132 | 0.89 | 10/10 |
| ylphenyl)-4,5,6,7- | | | | | | | | | | |
| tetrahydro-1,3- | | | | | | | | | | |
| benzothiazol-7-ol (20 | | | | | | | | | | |
| PO) | | | | | | | | | | |
| Vincristine (0.8 IP) | 1.33 | 80 | 0.01 | 2.14 | 43 | <0.01 | 5.29 | 58 | 0.13 | 9/10 |

a) Examples 5-7 and 2-(4-pyrrolidiny-1-ylphenyl)-4,5,6,7-tetrahydro-1,3-benzothiazol-7-ol administered on days 1 through 20. Vincristine administered on days 1, 5 and 9.
b) Relative Tumor Growth = Mean Tumor Mass on days 7, 14 and 21/Mean Tumor Mass on Day 0
c) % T/C = Relative Tumor Growth of Treated Group/Relative Tumor Growth of Placebo Group × 100
d) Statistical Analysis (Student's T-test) of Log Relative Tumor Growth. A p-value (p ≦ 0.05) indicates a statistically significant reduction in Relative Tumor Growth of Treated Group compared to the Placebo Control.
e) S/T = # of Survivors/# of Treated on Day +21 post tumor staging

What is claimed is:

1. A compounds of formula I: 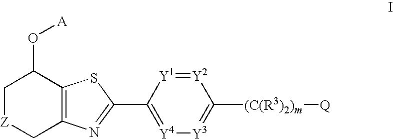
and pharmaceutically acceptable salts thereof,
wherein
A is H, C₁-C₃ alkyl, or acetyl;
Z is NR³ or O;
Q is —NR¹R², —NR³N(R³)₂, —NR³OR³, or —OH;
R¹ and R² are each independently H or C₁-C₃ alkyl, or R¹ and R² join together with the nitrogen atom to which each is attached, forming a 4 to 6 membered saturated heterocyclic ring comprising heteroatoms selected from 1-2 nitrogen atoms, 0-1 oxygen atom and 0-1 sulfur atom, said ring optionally substituted with one or more of R⁴;
R³ at each occurrence is independently H or C₁-C₃ alkyl;
R⁴ is C₁-C₃ alkyl, —N(R³)₂, or —OH;
Y¹, Y², Y³, and Y⁴ are the same or different, and are each independently N or CR⁵, or two R⁵ groups on adjacent carbon atoms join together, with the carbon atoms which they are bonded, to form a 9 to 10 membered bicyclic aryl ring or bicyclic heteroaryl ring, said ring comprising members selected from CR⁵ and N;
R⁵ is independently H or is independently selected from C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, where Ar is phenyl, 2-pyridyl, 3-pyridyl, 4-pyridyl, 2-thienyl, 3-thienyl, 2-furyl, or 3-furyl, optionally substituted with one or more of R⁴;
R⁶ is independently H or is independently selected from C₁-C₈ alkyl, C₂-C₈ alkenyl, and C₂-C₈ alkynyl, each optionally substituted with —NR¹R², —OR³, C₄-C₆ cycloalkyl, a saturated heterocyclic ring comprising heteroatoms selected from 0-1 nitrogen atom, 0-1 oxygen atom and 0-1 sulfur atom, or —COCH₃, C₄-C₆ cycloalkyl, optionally substituted with R⁴; or a saturated heterocyclic ring comprising heteroatoms selected from 0-1 nitrogen atom, 0-1 oxygen atom and 0-1 sulfur atom, and optionally substituted with one or more of R⁴; and
m is 0 or 1.

2. The compound of claim 1, wherein m is 0, A is H and Z is NR³.

3. The compound of claim 1, wherein m is 0, A is H and Z is O.

4. The compound of claim 1, wherein m is 0, A is H and Z is NH.

5. The compound of one of claims 1-4, wherein the ring formed by Y¹, Y², Y³, and Y⁴ is selected from: phenyl, pyridinyl, pyrimidinyl, and pyrazinyl.

6. The compound of one of claims 1-4, wherein Y¹ and Y² or Y³ and Y⁴ are CR⁵ and the two R⁵ groups on adjacent carbon atoms join together, with the carbon atoms which they are bonded, to form a 9 to 10 membered bicyclic aryl ring or bicyclic heteroaryl ring selected from: napthyl, indenyl, indolyl, benzoxazolyl, benzothiazolyl, benzofuranyl, benzisoxazolyl, benzimidazolyl, N-methylbenzimidazolyl, azabenzimidazolyl, indazolyl, quinazolinyl, quinolinyl and isoquinolinyl.

7. A compound selected from: 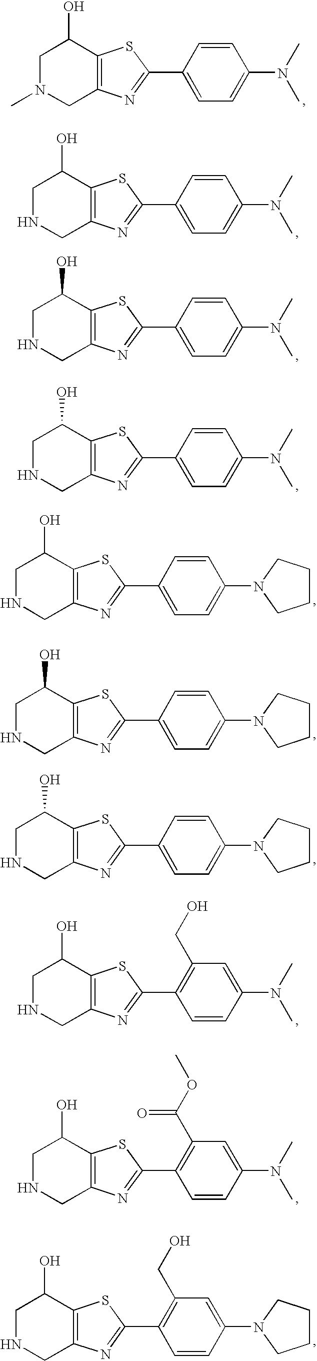 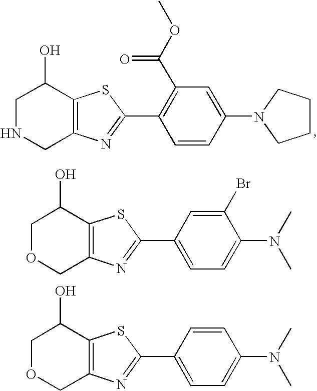
and pharmaceutically acceptable salts thereof.

8. A compound selected from: 2-(4-dimethylamino-phenyl)-5-methyl-4,5,6,7-tetrahydro[1,3]thiazolo[4,5-c]pyridine-7-ol, (R,S)-2-(4-dimethylamino-phenyl)-4,5,6,7-tetrahydro[1,3]thiazolo[4,5-c]pyridin-7-ol, (R)-2-(4-dimethylamino-phenyl)-4,5,6,7-tetrahydro[1,3]thiazolo[4,5-c]pyridin-7-ol, (S)-2-(4-dimethylamino-phenyl)-4,5,6,7-tetrahydro[1,3]thiazolo[4,5-c]pyridin-7-ol, (7R,S)-2-(4-pyrrolidin-1-yl-phenyl)-4,5,6,7-tetrahydro[1,3]thiazolo[4,5-c]pyridin-7-ol, (7R)-2-(4-pyrrolidin-1-yl-phenyl)-4,5,6,7-tetrahydro[1,3]thiazolo[4,5-c]pyridin-7-ol, (7S)-2-(4-pyrrolidin-1-yl-phenyl)-4,5,6,7-tetrahydro[1,3]thiazolo[4,5-c]pyridin-7-ol, 2-(2-hydroxymethyl-4-dimethylamino-phenyl)-4,5,6,7-tetrahydro[1,3]thiazolo[4,5-c]pyridine-7-ol, methyl 5-(dimethylamino)-2-(7-hydroxy-4,5,6,7-tetrahydro[1,3]thiazolo[4,5-c]pyridine-2-yl)benzoate, (7R,7S)-2-(2-hydroxymethyl-4-pyrrolidin-1-yl-phenyl)-4,5,6,7-tetrahydro[1,3]thiazolo[4,5-c]pyridin-7-ol, (7R)-2-(2-hydroxymethyl-4-pyrrolidin-1-yl-phenyl)-4,5,6,7-tetrahydro[1,3]thiazolo[4,5-c]pyridin-7-ol, (7R,7S)-2-(2-hydroxymethyl-4-pyrrolidin-1-yl-phenyl)-4,5,6,7-tetrahydro[1,3]thiazolo[4,5-c]pyridin-7-ol, methyl 5-(4-pyrrolidin-1-yl)-2-(7-hydroxy-4,5,6,7-tetrahydro[1,3]thiazolo[4,5-c]pyridin-2-yl)benzoate, 2-(2-amino-4-pyrrolidin-1-yl-phenyl)-4,5,6,7-tetrahydro[1,3]thiazolo[4,5-c]pyridin-7-ol, 2-(2-nitro-4-pyrrolidin-1-yl-phenyl)-4,5,6,7-tetrahydro[1,3]thiazolo[4,5-c]pyridin-7-ol, 2-[3-bromo-4-(dimethylamino)phenyl]-6,7-dihydro-4H-pyrano[3,4-d][1,3]thiazol-7-ol, 2-[4-(dimethylamino)phenyl]-6,7-dihydro-4H-pyrano[3,4-d][1,3]thiazol-7-ol and pharmaceutically acceptable salts thereof.

9. A pharmaceutically acceptable ester of a compound of claims 1-8.

10. A pharmaceutical composition comprising a compound of one of claims 1-9 and a pharmaceutically acceptable carrier.

11. A method for preparing a compound of claim 1, comprising the steps of:
(a) reacting a compound of formula 2: 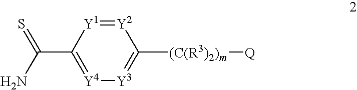
with tert-butyl 5-oxo-7-oxa-3-azabicyclo[4.1.0]heptane-3-carboxylate, thereby forming a compound of formula 3: 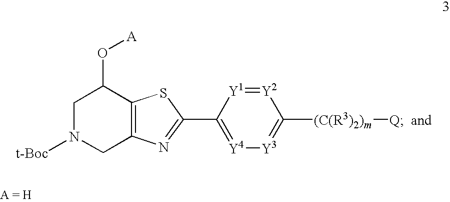
(b) treating the compound of formula 3 formed in step (a) with an acid,
wherein A is H.

12. A method for preparing a compound of claim 1, comprising the steps of:
(a) cyclizing a compound of formula 8: 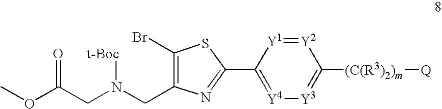
thereby forming a compound of formula 9: 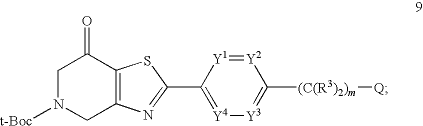
(b) treating the compound of formula 9 formed in step (f) with a reducing agent (sodium borohydride), thereby forming a compound of formula 10: 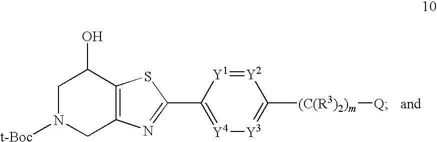
(c) treating the compound of formula 10 formed in step (b) with a deprotecting agent.

13. A method for preparing a compound of claim 1, comprising the steps of:
(a) cyclizing a compound of formula 7: 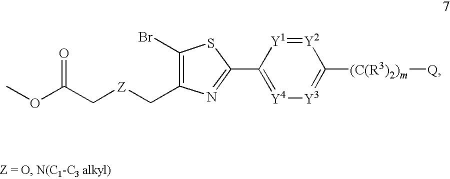
thereby forming a compound of formula 9: 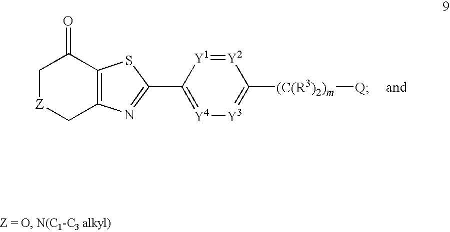
(b) treating the compound of formula 9 formed in step (a) with a reducing agent.

14. A method for treating a disease associated with securin comprising administering to a subject in need a therapeutically effective amount of a compound of any one of claims 1-9.

15. The method of claim 14, wherein the therapeutically effective amount is from 0.1 mg/kg/day to 1000 mg/kg/day.

16. The method of claim 14 or 15, wherein the disease associated with securin is a cancer selected from: breast, colon, lung, prostate, melanoma, epidermal, leukemia, kidney, bladder, mouth, larynx, esophagus, stomach, ovary, pancreas, liver, skin, thyroid, prostate and brain cancer.

17. The method of claim 16, wherein the cancer is breast cancer.

18. A method for inhibiting tumor growth in a subject comprising administering to a subject in need a therapeutically effective amount of a compound any one of claims 1-9.

19. The method of claim 18 or 19, wherein tumor growth is associated with a cancer selected from: breast, colon, lung, prostate, melanoma, epidermal, leukemia, kidney, bladder, mouth, larynx, esophagus, stomach, ovary, pancreas, liver, skin, thyroid, prostate and brain cancer.

20. The method of claim 18, wherein the therapeutically effective amount is from 0.1 mg/kg/day to 1000 mg/kg/day.

21. The method of claim 18, wherein tumor growth is associated with a human breast carcinoma.

22. The method of any one of claims 14-21, wherein a chemotherapeutic agent is used in combination with the therapeutically effective amount of a compound of claim 1.

23. A pharmaceutical composition comprising a compound of any one of claims 1-9 in combination with another kinase-inhibiting compound or chemotherapeutic agent, and a pharmaceutically acceptable carrier.
